(19) United States
(12) Patent Application Publication
(10) Pub. No.: US 2007/0122389 A1
(43) Pub. Date: May 31, 2007

(54) Stage specific follicle maturation systems

(76) Inventors: Teresa Woodruff, Chicago, IL (US); Lonnie D. Shea, Chicago, IL (US)

Correspondence Address: TODD S. PARKHURST;HOLLAND & KNIGHT LLP, 131 S. DEARBORN STREET, 30TH FLOOR, CHICAGO, IL, 60603, US

(21) Appl. No.: 11/642,118

(22) Filed: Dec. 20, 2006

Related U.S. Application Data
(63) Continuation-in-part of application No. 11/480,691, filed on Jul. 3, 2006.
(60) Provisional application No. 60/752,240, filed on Dec. 20, 2005.
(60) Provisional application No. 60/740,746, filed on Nov. 30, 2005.
(60) Provisional application No. 60/697,593, filed on Jul. 7, 2005.
(60) Provisional application No. 60/697,725, filed on Jul. 8, 2005.

(51) Int. Cl. A61K 35/12; C12N 5/06; C12N 5/08

(52) U.S. Cl. 424/93.7; 435/354; 435/366

(57) ABSTRACT

A three-dimensional matrix system is described herein. The system is used to surround developing tissue and support continued interaction between, for example, an oocyte and a supporting cellular structure. Oocytes grown to maturity can then be retrieved from the matrix for subsequent research use and/or fertilization. The systems and methods of this invention demonstrate normal cellular arrangement and oocyte growth during in vitro culture, with harvested oocytes competent for meiotic division and further maturation and development.

### CROSS REFERENCE TO RELATED APPLICATIONS

This application claims the benefit of U.S. Provisional Application 60/752,240 filed on Dec. 20, 2005. This application is a continuation-in-part of application Ser. No. 11/480,691 filed on Jul. 3, 2006 which claims the benefit of U.S. Provisional Application 60/697,593 filed on Jul. 7, 2005, U.S. Provisional Application 60/697,725 filed on Jul. 8, 2005, and U.S. Provisional Application 60/740,746 filed on Nov. 30, 2005.

### GOVERNMENT RIGHTS

This work was supported by National Institutes of Health Grant U54 HD41857. The U.S. Government may have certain rights in this invention.

### BACKGROUND OF THE INVENTION

Follicle cell maturation is a complex, multistage process that involves multiple cell types, cell-cell and cell-substrate interactions, and a variety of soluble stimuli (e.g. hormones and growth factors). “Folliculogenesis” can be divided into two phases: (1) preantral phase and (2) antral phase. Three major stages define the preantral phase of folliculogenesis: the primordial follicle stage, the primary follicle stage, and the secondary follicle stage. The development of a primordial follicle to a secondary follicle in humans can take ˜290 days and is characterized by growth and differentiation of the oocyte. The antral phase is regulated by follicle stimulating hormone, luteinizing hormone, and other growth factors. These phases are often divided according to (a) the small follicle stage (class 2, 3, 4, and 5), (b) the medium follicle stage (class 6), (c) the large follicle stage (class 7), and (d) the preovulatory follicle stage (class 8). The human ovaries produce a single dominant follicle (selected from class 5 follicles) that originates from the primordial follicle. Primordial follicles consist of an immature oocyte surrounded by a single layer of granulosa cells.

During the maturation of primary and secondary follicles (preantral), the oocyte increases in volume and the granulosa cells multiply to form several layers. To complete the follicle unit, thecal cells from the surrounding stroma differentiate to form a cell layer outside the granulosa cells. Oocyte growth is dependent upon gap junction mediated communication between the oocyte and its companion granulosa cells; the rate of growth is related to the number of granulosa cells coupled to the oocyte. These primary and secondary follicles then gradually progress to the next stage, following stimulation by growth and differentiation factors and then by pituitary hormones, follicle stimulating hormone (FSH) and luteinizing hormone (LH). FSH acts on a small number of follicles, causing them to begin explosive growth leading to a fully mature follicle. At the end of maturation, gonadotropin surges stimulate two events: oocyte maturation and cumulus expansion. Oocyte maturation involves progression from prophase of the first meiotic division to metaphase of the second meiotic division. The first indication of the resumption of meiosis is germinal vesicle breakdown (GVBD). Cumulus expansion, resulting from the gonadotropin surges, involves secretion of a hyaluronic acid-rich proteoglycan matrix. Ultimately, the dominant follicle expels the oocyte in a process known as ovulation. If the oocyte is not fertilized, new sets of follicles are recruited, and the cycle of follicular maturation and hormone activation continues.

The follicle is a three-dimensional structure and current culture methods on two-dimensional membranes or tissue culture plates do not maintain the requisite physiologic spatial arrangement of cells. Enzymatically isolated granulosa-enclosed oocytes grow on “stalks” above the membrane surface. Once the basal lamina surrounding the follicle is disrupted, granulosa cells in culture migrate away from the oocyte and onto available surfaces. Some three-dimensional systems based on collagen have been developed for culturing granulosa-oocyte complexes both in vitro and form implantation into kidney capsules. However, the collagen gel is not easily manipulated for studying individual follicles on a large scale and removal of the follicle following culture is difficult. Manually dissected follicles that retain all cell types including the stroma, and theca cells layers fare better than enzymatically obtained GOCs; the integrity of the follicle can be maintained if grown in conditions that do not allow attachment to the culture surface. However, manual dissection is labor-intensive and produces fewer follicles than enzymatic digestion.

The complex follicle developmental process is driven, in large part, by the follicle's interaction with local regulatory factors and endocrine signals. Follicle stimulating hormone (FSH) plays a critical role in this process, regulating estradiol secretion, development of antral follicles, and selection of the dominant follicle. Additionally, FSH is widely employed in assisted reproduction technologies to recruit supernumerary follicles for oocyte collection and for in vitro maturation of immature oocytes. In vivo treatment of mice with FSH resulted in retraction of transzonal projections and improved oocyte meiotic competence; however, high doses of FSH for in vitro maturation negatively impacted gamete quality. Many fundamental questions remain regarding the role of FSH in follicle and oocyte development, including the precise role of FSH in early follicle development and mechanism of action at successive stages of development. Although follicles are able to progress to the preantral stage in the absence of FSH-β or the FSH receptor, FSH levels are elevated during the first 10 days of life in female mice, which corresponds to a period of rapid follicle growth and development.

In vitro systems have been developed to better understand the complex mechanisms that regulate follicle maturation. These systems have been developed for a variety of species, including bovine, rat, and non-human primates, with the majority of these efforts centered on the development of systems for mouse follicle culture. FSH is a central component in such systems, but there are conflicting interpretations regarding the appropriate dosage and timing of FSH presentation. It is difficult to directly compare these different culture systems, due to differences in isolation and culture conditions. However, the dependence of follicle development on FSH may depend upon the stage of the follicle in the culture system. Although most studies have been restricted to examining a particular stage and not comparing different stages, FSH appears to be critical for continued development of late preantral follicles or early antral follicles. The exact role of FSH in earlier follicle development is less clear: two-layer secondary follicles isolated from immature mice do not respond to FSH alone, while two-layer secondary follicles isolated from adult mice grow larger in response to FSH. Additional studies demonstrated that 8-Br-cAMP or forskolin, but not FSH, could stimulate two-layer secondary follicles isolated from immature mice to grow in serum-free culture. However, in serum-supplemented cultures of two-layer secondary follicles isolated from immature mice, FSH was critical for follicle survival, growth, and antrum formation. In addition to the possible effect of FSH on different stages of follicles, the dose of FSH may impact follicle maturation. For example, early reports of in vitro cultured two-layer secondary follicles used a dose of 100 mIU/mL FSH to promote follicle survival and oocyte maturation, but a dose of 10 mIU/mL FSH was later reported to be the minimal dose required for oocytes in these cultured follicles to obtain meiotic competence. In a study of multilayer secondary follicles, a dose of 100 mIU/mL FSH produced the maximum rate of growth, but estradiol secretion was significantly higher with increased doses of FSH.

One potential limitation of these systems is the disruption of follicle architecture that can occur when follicles are cultured on a two-dimensional substrate. The change in the follicle morphology may alter the paracrine signaling that is critical to follicle maturation, as the altered cell-cell organization could result in diffusion of paracrine signals away from the target cells. Additionally, in vivo the inner layers of granulosa cells are not directly exposed to endocrine signals due to the exclusion of the vascular system by the basal lamina, while in the disrupted architecture of two-dimensional systems there are few granulosa cell layers between the oocyte and the media.

What is needed is a method to maintain the cell-cell organization while coordinating the level of FSH in a culture system with the developmental stage of the follicle for appropriate granulosa cell proliferation and differentiation, and for production of healthy oocytes. Furthermore, a systematic study of FSH in a polysaccharide-based hydrogel culture system is needed. One such polysaccharide is alginate. Alginate, for example, is a linear polysaccharide derived from algae and composed of repeating units of β-mannuronic acid and α-L-guluronic acid. It gels by ionic cross-linking of the guluronic residues. This mild gelation process maintains cell viability. Additionally, granulosa cells do not interact with alginate, allowing intact follicles to be retrieved from the matrix for in vitro maturation of the oocyte.

What is also needed is an in vitro system that optimizes growth and/or maturation of specific stage follicles. For example, an in vitro system that optimizes preantral two-layer secondary follicle growth and maturation, or preantral multilayer secondary follicle growth and maturation, or oocyte developmental competence is needed. Such a system will center on hydrogels, wherein the mechanical properties of the hydrogel can be manipulated to allow greater follicle expansion.

### BRIEF SUMMARY OF THE INVENTION

It is an object of the present invention to provide one or more three-dimensional matrix culture systems and/or related methods of use for the in vitro maturation of germ line cells, thereby overcoming various deficiencies and short-comings of the prior art. A three-dimensional matrix system may be used to surround developing tissue and support continued interaction between, for example, an oocyte and a supporting cellular structure. Oocytes grown to maturity can then be retrieved from the matrix for subsequent research use and/or fertilization. The systems and methods of this invention demonstrate normal cellular arrangement and oocyte growth during in vitro culture, with harvested oocytes competent for meiotic division and further maturation and development. For example, pre-antral two-layer secondary and pre-antral multilayer secondary follicles can be cultured in alginate-based matrices with increasing doses of recombinant human FSH. As shown below, the effects of FSH dose on follicle survival, growth, metabolism, steroid production, and oocyte development have been measured using the present invention.

It is another object of the present invention to provide an in vitro follicle culture system having alginate-based matrices modified with extracellular matrix (ECM) molecules, proteins, and/or peptides. ECM composition is known to affect granulosa cell differentiation in vitro. For example, a synthetic matrix composed of alginate, modified with peptides comprising the RGD amino acid sequence, supports granulosa cell adhesion and spreading, and increased estradiol and progesterone secretion. The present invention also provides a method for regulating follicle development in vitro based upon the ECM identity and the stage of follicle development. Extracellular matrix molecules, proteins, and/or peptides include, but are not limited to, the tri-amino acid peptide “RGD”, other proteins and peptides having the tri-amino acid sequence “RGD”, the peptide YIGSR, the peptide GGGGRGDS, and the peptide IKVAV. These proteins and peptides may be linked to the matrix by reacting the amino group on the peptide or protein with the carboxylic acid on an alginate molecule.

It is still another object of the present invention to provide an in vitro follicle culture system which can be adapted to the different maturation stages of follicle development. The developmental requirements of ovarian follicles are dependent upon the maturation stage of the follicle. For example, in vitro, and as further described below, preantral two-layered follicles survive but do not grow in the absence of FSH. Preantral multi-layered follicles will die in the absence of FSH.

The present invention provides a novel system for growing and maturing cells and tissue including, but not limited to ovarian follicles containing oocytes, by providing a novel, synthetic, three-dimensional scaffold that can be used for the encapsulation and subsequent culture of cells and tissue including but not limited to immature follicles. The herein described novel three-dimensional scaffold is an improvement over prior art 2-dimensional scaffolds and prior art “sandwich” embedding gel structures in that it better maintains the organization of encapsulated cells, for instance, those cells within the follicle complex (i.e., oocyte and any associated granulosa cells). In the case of oocytes, the 2-dimensional surfaces utilized in most current approaches may result in a disruption in the interaction between the oocyte and the granulosa cells, and this disruption may negatively impact the growth and maturation of the oocyte. Furthermore, in the case of “sandwich” embedding gel structures, wherein a cell to be grown is inserted between two pre-formed gel beds, the existence of fault lines between the preformed gel slabs allow for open channels which connect the follicle to the outside of the gel gel sandwich. Such sandwich structures do not allow for complete engulfment of the cells, tissue, or follicle cells to be developed, matured, or grown. The present invention can be used to overcome these disadvantages. The present invention provides for a proximal gel matrix environment at all positions around the periphery of the follicle cells, cells, or tissue. The present invention is directed to, for example, an in vitro method for maturing a preantral follicle comprising (a) suspending a preantral follicle into a non-crosslinked alginate solution, wherein the solution comprises less than 2% alginate weight per volume; (b) crosslinking the suspension, thereby forming a preantral follicle-three dimensional gel matrix; (c) culturing the preantral follicle in the three dimensional matrix, wherein the preantral follicle forms an antral cavity and whereby a cumulus-oocyte complex is formed; and (d) releasing the antral follicle from the three dimensional gel matrix. Optionally, the foregoing method may further comprise (e) culturing the released antral follicle in culture media comprising one or more pituitary hormones, wherein polar bodies are formed; and (f) releasing the oocyte from the antral follicle.

In yet another option, steps (e) and (f) in the above-described embodiment of the present invention, may be replaced with (e) isolating the cumulus-oocyte complex from the antral follicle; (f) culturing the isolated cumulus-oocyte complex in culture media comprising one or more pituitary hormones, wherein polar bodies are formed; and (g) releasing the oocyte from the cumulus-oocyte complex.

In yet another alternative to step (g) in the foregoing embodiment of the present invention, one may (g) remove the cumulus-oocyte complex from the culture.

In yet another embodiment of the present invention, an in vitro method for maturing a preantral follicle is provided, comprising (a) suspending a preantral follicle into a non-crosslinked alginate solution, wherein the solution comprises less than 2% alginate weight per volume; (b) crosslinking the suspension, thereby forming a preantral follicle-three dimensional gel matrix; (c) culturing the preantral follicle in the three dimensional matrix, wherein the preantral follicle forms an antral cavity and whereby a cumulus-oocyte complex is formed; (d) releasing the antral follicle from the three dimensional gel matrix; (e) culturing the released antral follicle in culture media comprising one or more pituitary hormones, wherein polar bodies are formed; and (f) isolating the cumulus oocyte complex from the cultured antral follicle.

It is well known that embryonic stem cells can be isolated from blastocysts produced from a fertilized oocyte or ovum. Disclosed herein are methods for producing embryonic stem cells and stem cell lines. These methods are based upon the present findings that a blastocyst can be obtained from a fertilized, matured oocyte, wherein the matured oocyte is produced from the in vitro preantral follicle maturation methods described herein.

As used herein, the terms “scaffold” and “matrix” are used interchangeably and represent a material that is used to contact or support a follicle in a three-dimensional manner wherein there is a proximal gel matrix environment at all positions around the periphery of the follicle cells, cells, or tissue. The scaffold or matrix can be covalently modified with saccharides, proteins, peptides, or nucleic acids. Covalent modification may be accomplished prior to crosslinking the scaffold or matrix; or subsequent to crosslinking the scaffold or matrix. The gelled, or crosslinked, matrix provides a support to the follicle complex, maintains the organization of the cells, allows for diffusion of various growth factors through the support, and generally provides an environment conducive to maturation. Examples of material that can be used as solid substrates include peptide polymers, peptoid polymers, polysaccharides, carbohydrates, hydrophobic polymers, and amphiphilic polymers. Examples of polysaccharide include, but are not limited to, alginate and hyaluronic acid. Furthermore, it is to be understood that there are other examples of hydrogels which are familiar to one of ordinary skill in the art; such as, polyacrylamide, PEG hydrogels, and NIPAM. There are many cross-linking agents known to one of ordinary skill in the art. For example, calcium chloride, magnesium chloride, barium sulfate, and any divalent cations may be used to crosslink many solutions. Examples of growth factors include, but are not limited to, inhibins, activins, selenites, and transferrins. Examples of hormones included, but are not limited to follicle stimulating hormone and luteinizing hormone.

As used herein, and as is well known in the art, the term “follicle maturation” is distinguished from “follicle growth” in that “follicle maturation” is directed to the formation of new physical characteristics or the formation of distinct morphological markers, such as an antral cavity; or cumulus cells around the oocyte; or granulosa cell proliferation and differentiation; or theca cell proliferation and differentiation; or steroid production. “Follicle growth” is directed to an increase in size of the follicle cell. “Follicle cells” can encompass the oocyte, granulosa cells, and/or theca cells. “Oocyte growth” (an increase in size) is distinguished from “oocyte maturation” (developing a greater capacity to resume meiosis). “Follicle expansion” refers to a change in size of the follicle within a hydrogel, or hydrogel bead, wherein there is outward pressure from within the bead. For example, a bead with increased mechanical properties (for example, increased alginate percent (weight per volume)) will limit expansion more than a bead with decreased mechanical properties.

As used herein, the term “two-layered secondary follicle” refers to a preantral follicle comprising an oocyte surrounded by 2 layers of granulosa cells. A “multilayered secondary follicle” refers to a preantral follicle comprising an oocyte surrounded by more than 2 layers of granulocytes.

The systems and related methods of the present invention provide matrix materials affording incorporated cellular matter, 3-dimensional support and/or contact sufficient to promote desired physiological growth and development—such contact and/or support as may be provided at least in part by the matrix material, a modification thereof and/or such material or modified material in association with a growth factor, hormone, serum protein or any such other culture additive. Cellular matter can be introduced or incorporated as described below in the context of an alginate matrix material, by encapsulation or bead formation with subsequent gelation. Alternatively, this invention contemplates various other procedures known for introduction of cellular matter into matrix materials, together with techniques for subsequent culture, growth and/or maturation of the cellular matter.

Likewise, such a system can optionally include at least one cytokine, growth factor and/or serum protein. Various combinations of growth factors and other hormones or additives can be determined, as understood by those skilled in the art, without undue experimentation. Such combinations could, for example, include insulin, various growth factors, follicle-stimulating hormones, luteinizing hormones, inhibin and activin, among other such additives. However, as will be apparent from the following examples and data, such additives are not necessarily required in conjunction with the systems and related methods of this invention.

In part, the present invention also comprises a kit capable of assembly for a culture, growth and/or development of mammalian germ cells. Such a component kit comprises a matrix material; and at least one culture additive selected from known cell growth factors, hormones and nutrients. As discussed elsewhere herein, a component matrix material can comprise a gelable polymer. Various embodiments of such a kit may comprise a polysaccharide, with the matrix material further comprising a suitable cross-linking agent. Without limitation, such a polysaccharide can be provided as a dry powder, with a corresponding cross-linking agent comprising a calcium salt. Alternatively, several kit embodiments may comprise a matrix precursor material and an agent or component for material coupling or cross-linking and resultant 3-dimensional matrix formation. Optionally, the component matrix material of such a kit can be modified as described elsewhere herein to enhance cell-matrix interactions and/or signaling. Further, a kit of this invention can further comprise one or more culture additives such as but not limited to a variety of cytokines, growth factors, serum proteins, hormones and nutrients. Such additives may be provided for incorporation into the component matrix or, alternatively, in solution for subsequent introduction.

A kit of this invention can further include hardware and/or equipment for suspending cellular matter in the matrix material and/or introduction of such matter and/or matrix with a coupling or cross-linking agent. Various other kit components can be provided, as would be known to those skilled in the art, such components including but not limited to micromanipulators for transferring matrix/cell material, diagnostic reagents for determining stage or extent of cell growth or maturation, and reagents for matrix release and recovery of the cellular matter.

Several such embodiments comprise maintenance of the granulosa cells with the oocyte, large-scale preparation by enzymatic digestion of the ovary, and serum-free growth conditions, with the ability to directly and to easily study individual granulosa cell-oocyte complexes during in vitro development. In one such system, individual mouse granulosa cell-oocyte complexes (GOCs) were incorporated into a three-dimensional culture system prepared using an alginate material, then tested for the ability to produce mature oocytes: immature ovarian granulosa-oocyte complexes can be matured in a three-dimensional alginate matrix without the addition of serum to produce viable oocytes capable of resuming meiosis.

### BRIEF DESCRIPTION OF THE DRAWINGS

FIG. 1. Follicle encapsulation in alginate based gels. A. Follicles (100-130 μm or 150-180 μm) are mechanically isolated with basement membrane and theca/stroma cells attached. B. Individual follicles are pipetted into alginate drops on a mesh screen. The mesh screen may be made of polypropylene, teflon, or stainless steel, for example. C. After all follicles are transferred, the mesh is immersed in 50 mM CaCl₂ to cross-link the gel and encapsulate the follicle within the matrix.

FIG. 2. To examine incorporation and retention of ECM within alginate beads, collagen I was iodinated using a Bolton-Hunter iodination protocol (41). Alginate-I¹²⁵-Collagen I beads were made and kept in αMEM supplemented with 1% penicillin-streptomycin at 37° C. for 8 days. Every other day, the media was exchanged for fresh media and both the beads and removed media were analyzed on the gamma counter to determine the amount of collagen present in the bead over time. A. Alginate-ECM beads formed with I¹²⁵-labelled collagen I show a linear increase in counts with increasing number of beads (R=0.999), indicating uniform distribution of the collagen I within the alginate gel. B. The incorporated ECM slowly diffuses from the alginate gels, such that 83.5±1.6% of the ECM is maintained over the 8 day culture period. Data presented as average±SD of 3 replicates.

FIG. 3. A. Secondary follicle size increased when cultured in Cl or RGD matrices compared to ALG. B. Only Cl, and RGD result in a significant increase compared to ALG. C-E. A representative secondary follicle on day 0 of culture (C) and on day 8 after culture in ALG (D) or Cl (E). Ooc=oocyte, scale bar=25 urn. Significant differences are denoted by different letters, p<0.05.

FIG. 4. A. Preantral follicles cultured in FN or LN matrices grow significantly less than follicles cultured in either ALG or Cl matrices. B. FN, CIV, and LN all result in a significant decrease compared to ALG.

FIG. 5. Preantral follicle steroid secretion profiles in different matrices. Culture in ECM matrices significantly increases secretion of progesterone (A) and decreases secretion of 17£-estradiol (B) compared to culture in ALG by day 8 in culture. * indicates significantly different than all other conditions, p<0.05.

FIG. 6. A. Preantral follicles cultured in different matrices have different meiotic competency rates.

FIG. 7. Two-layer secondary follicle growth and metabolism in alginate-collagen I gels. A. Two-layer secondary follicles cultured with increased levels of FSH grew significantly larger. B. The increase in FSH dosage resulted in a significant increase in lactate accumulation on day 8 of culture. Data represented as average±SEM, number examined for each condition given in Table 1. Points or bars without common superscripts differ significantly between treatments for isolated time points, p<0.05.

FIG. 8. Two-layer secondary follicle steroid secretion in alginate-collagen I gels. (A) Two-layer secondary follicles cultured with 10 or 25 mIU/mL FSH secreted significantly more progesterone at the end of culture compared to day 2 (p<0.05); progesterone levels were not significantly different in response to increased doses of FSH. (B) Two-layer secondary follicles cultured with 5, 10, or 25 mIU/mL FSH secreted significantly more 17β-estradiol at the end of culture compared to day 2 (p<0.05). Increased levels of FSH also resulted in a significant increase in 17β-estradiol at the end of the culture. Data represented as average±SEM, n=3. Points without common superscripts differ significantly between treatments for isolated time points, p<0.05. Follicles cultured without FSH did not secrete detectable levels of 17β-estradiol or progesterone.

FIG. 9. Multilayer secondary follicle growth and metabolism in alginate gels. A. Multilayer secondary follicles cultured with increased levels of FSH grew significantly larger. Data represented as average±SEM, number examined for each condition given in Table 1. B. The increase in FSH dosage resulted in a significant increase in lactate accumulation on day 8 of culture. Data represented as average±SEM, n=3. C. Multilayer secondary follicles cultured with 50 mIU/mL FSH incorporated more 3H-thymidine from the second to third day of culture. Data represented as average±SEM, n=5. Points or bars without common superscripts differ significantly between treatments for isolated time points, p<0.05.

FIG. 10. Multilayer secondary follicle steroid secretion in alginate gels A. Progesterone was increased significantly at the highest dose of FSH examined. Follicles cultured without FSH did not secrete detectable levels of progesterone. B. 17£-estradiol increased significantly with time for multilayer secondary follicles cultured with FSH supplemented media. Increased levels of FSH also resulted in significant differences in 17£-estradiol levels. Data represented as average+SEM, n=3. Points without common superscripts differ significantly between treatments for isolated time points, p<0.05.

FIG. 11. In vitro matured oocytes isolated from multilayer secondary follicles cultured in alginate gels. A. Oocytes from follicles cultured in 50 mIU/mL FSH were not significantly different in size compared to in vitro or in vivo controls. Data represented as average±SEM, number examined for each condition given in Table 2. Points without common superscripts differ significantly between treatments, p<0.05.

FIG. 12. Two-layered secondary follicle growth in alginate scaffold of different concentrations. During the first 6 days of culture, no significant difference in follicle size was observed among the four groups. At day 8, follicles encapsulated in 1.5% alginate demonstrated significantly less growth than those in the other three alginate concentrations. At day 10 and 12, follicles in 0.5% and 0.25% and 0.5% alginates were significantly larger than follicles grown in 1.5% and 1.0% alginate. Different letters within each platform indicate statistically significant differences (p<0.05).

FIG. 13. Steroid secretion profiles of two-layered secondary follicle cultured in alginate of different concentrations. All steroid levels increased significantly from baseline over time. From day 8 to day 12, follicles grown in 1.5% alginate showed significantly decreased androstenedione secretion (A). No significant changes in estradiol secretion between alginate conditions was observed at day 8, but from days 10-12, estradiol secretion by follicles cultured in 1.5% alginate was significantly less than the estradiol secreted by follicles in the other alginate conditions (B). Progesterone secretion was significantly lower in follicles encapsulated in 0.5% and 0.25% alginate compared with those grown in 1.5% and 1.0% alginate through last six days culture (C). Data represented as average+/−SEM, n=4. Statistical significance was observed between groups with different letters (p<0.05).

FIG. 14. Relative gene expression levels characterized by real-time PCR. FSHR and C×43 expression was not significantly different in response to change of alginate concentration. LHR expression was significantly up-regulated in follicles encapsulated in 0.25% alginate. LHR expression in 0.25% alginate was 2-fold higher than those in 0.5% and 1.0% alginate, and 4-fold higher than 1.5% alginate (A). GDF9 and MATER expression were slightly lower in the 1.5% alginate group compared with the other groups, but the difference was not significant (B). Statistical significance was observed between groups with different letters (−<0.05).

FIG. 15. Oocyte size from different alginate concentration groups. On day 8, the average diameter of oocytes cultured in 0.025% alginate was smaller than those cultured in the other concentration alginates (A). However, on day 12, the average diameter of oocytes cultured in 1.5% alginate was smallest (B). Decreasing alginate concentration increased overall oocyte growth between day 8 and day 12 (C). Statistical significance was observed between groups with different letters (p<0.05).

FIG. 16. (A) Basal steroid concentrations were measured from the condition media collected every other day (A: androstenedione, E2: estradiol, P: progesterone). Data represented as mean+/−SE (n=4. Androstenedione increases represent theca cells maintaining normal physiologic function. Estradiol and progesterone both increased, and the ratio of P to E2 was less than 0.5. (B) Oocytes from in vitro cultured multilayered secondary follicles increased significantly from day 0 to day 8 (n=30; p<0.05). In additional, the final size of in vitro growth oocytes was not significantly different in size from oocytes grown in vivo (n=30; p=0.078). (C) Most fully grown oocytes (n=99) underwent germinal vesicle breakdown and progressed to metaphase II, although at slightly lower rates than oocytes developed in vivo (n=93). (D) The fertilization rate, determined by the appearance of 2 pronuclei, was 68.2%+/−14.5% for metaphase II oocytes (n=86) from in vitro culture follicles, and 81.7%+/−5.0% for metaphase II oocytes (n=65) from in vivo control. Statistical significance was noted between groups with different letters.

### DETAILED DESCRIPTION OF THE INVENTION

In general, the methods of the present invention have shown to be useful in the growth and/or maturation and/or fertilization of mammalian oocytes. Furthermore, the present invention relates to the production of embryonic stem cells which are useful for embryological studies, studies of diseases, clinical applications, experimental models, and the like on primates, particularly humans and monkeys. The present invention includes the use of two-layered secondary follicles (100-130 μm), containing oocytes from about 50 to 65 μm, and multilayered secondary follicles (150-180 μm). The basement membrane and theca cells may optionally be included in each oocyte complex to be matured. Each oocyte complex may be freshly prepared or prepared from a frozen environment. Each follicle is placed into an in vitro follicle culture system which can be adapted to the different maturation stages of the follicle's development. The developmental requirements of ovarian follicles are dependent upon the maturation stage of the follicle. For example, in the present invention, pre-antral multi-layered follicles require FSH for growth.

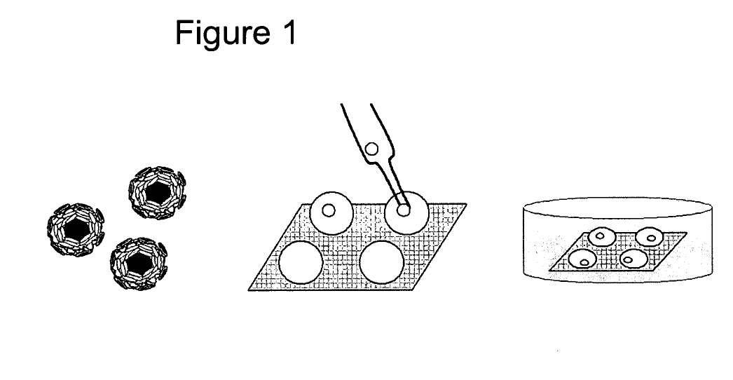

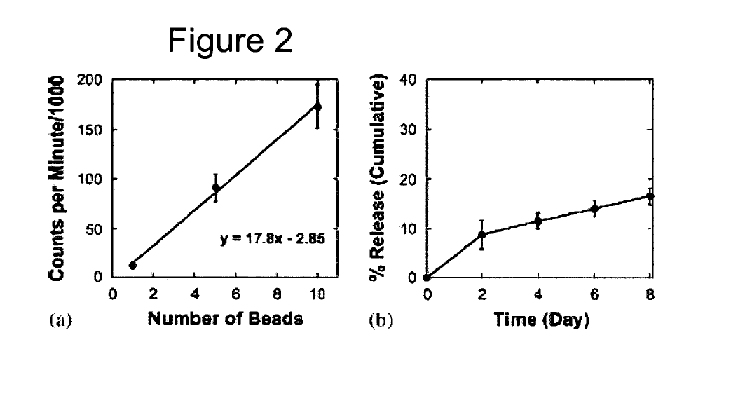

The herein described alginate-based hydrogels, which can be modified with either ECM molecules and/or proteins or peptides having an RGD sequence and/or peptides consisting of RGD, may be employed as a synthetic matrix to reconstitute the basement membrane and ovarian stroma for the three-dimensional culture of ovarian follicles in vitro. For example, the present invention may incorporate the use of fibronectin, collagen, laminin, peptides and proteins comprising the sequence GGGGRGD, and cyclic peptides comprising an RGD sequence. Immature follicles can be cultured within these alginate-ECM matrices, and maturation characterized by one or more of granulosa cell differentiation, antral cavity formation, and/or the meiotic competence of the oocyte. It is recognized herein that alginate can modified with, for example, ECM molecules or RGD containing peptides. The present invention allows for ECM molecules such as collagen Type I, fibronectin, laminin and collagen Type IV to be mixed with cross-linkable solutions of alginate of varying concentrations. Follicles are then encapsulated into the alginate-ECM matrix. For example, droplets of the alginate-ECM solution are suspended on, for example, a polypropylene mesh. A single follicle is pipetted into each droplet in a minimal amount of media (see FIG. 1). After all droplets have been filled, the mesh is immersed in sterile crosslinking solution, for example 50 mM CaCl₂ for 2 minutes to cross-link the alginate, and then rinsed, for example, in L-15 media. This procedure allows for a proximal gel matrix environment at all positions around the periphery of the follicle.

Follicle stimulating hormone (FSH) is a central component in many in vitro systems that have been developed to understand the complex mechanisms that regulate follicle maturation. FSH appears to be critical for continued development of late preantral follicles or early antral follicles. However, the exact role of FSH in earlier follicle development is less clear, two-layered secondary follicles isolated from immature mice do not respond to FSH alone; while two-layer secondary follicles isolated from adult mice grow larger in response to FSH.

The present invention centers on a novel three-dimensional culture system where individual immature mouse granulosa-oocyte complexes or intact follicles are encapsulated within alginate beads for culture. In this system, the alginate matrix provides a mechanical support for the follicle as it increases in size, allowing examination of the role of various factors in follicle maturation while maintaining an in vivo-like morphology. Additionally, encapsulating the follicle within a three-dimensional matrix allows for studies of how the interactions of the outer layers of somatic cells and insoluble factors such as the extra-cellular matrix direct follicle maturation. Using the present alginate-based matrix invention, it has been determined that the level of FSH in a culture system must be coordinated with the developmental stage of the follicle for appropriate granulosa cell proliferation and differentiation, and for the production of healthy oocytes.

Alginate, a linear polysaccharide derived from algae and composed of repeating units of β-mannuronic acid and α-L-guluronic acid, gels by ionic cross-linking of the guluronic residues. This mild gelation process maintains cell viability. Additionally, granulosa cells do not interact with alginate, allowing intact follicles to be retrieved from the matrix. As described in more detail below, two-layer and multilayer secondary follicles can be cultured in alginate-based matrices with increasing doses of recombinant human FSH. The below-identified examples show, by using the present invention, how one can culture two-layer secondary and multilayer secondary follicles in alginate-based matrices with increasing doses of FSH to determine the effect of FSH dose on follicle survival, growth, metabolism, steroid production, and oocyte development. Furthermore, the below-identified examples illustrate how the present invention optimizes the culture of two-layered and multilayered secondary follicles by coordinating the level of FSH with the developmental stage of the follicle.

The present invention also optimizes preantral two-layer secondary follicle growth and maturation, preantral multilayer secondary follicle growth and maturation, and oocyte developmental competence by encapsulating individual follicles into alginate beads of having optimal concentrations of alginate. Alginate beads can be fabricated with controlled mechanical properties and a range of diameters. Two key parameters that are herein shown to influence the mechanical properties are the final concentration of alginate and the concentration of calcium chloride. The present invention is directed to altering the mechanical properties of alginate matrices. These mechanical properties have been reduced to allow for greater follicle expansion. This expansion allows for the development of theca cells. Thus, beads can be fabricated, for example, at several alginate and calcium chloride concentrations. Previous results have shown that beads with defined shapes and reproducible properties are not optimally formed from alginate solutions less than 0.5% or with calcium chloride concentrations less than 25 mM. Furthermore, two-layered secondary follicles could not be matured with high efficiency in 2% alginate beads (data not shown). Until now, the mechanical properties of hydrogel matrices have not been studied as factors limiting in vitro follicle cell development. The below-identified examples show, that by lowering the mechanical properties of alginate (i.e. the weight per volume percentage of alginate in the hydrogel), theca and granulosa cell proliferation, follicle expansion, and follicle maturation is promoted. Using alginates as an example, the carboxylic acid residues along the polysaccharide backbone can be modified with adhesion peptides from extracellular matrix molecules to modulate the interaction of granulosa cells with the scaffold. The peptide sequence RGD, which is found in collagen type I and fibronectin; and YIGSR from laminin, for example can be coupled to alginate to modulate specific cellular adhesion. The length of the peptide sequence can be varied to affect cell adhesion. Glycine, for instance, can be used as a spacer molecule as it is uncharged and does not contain functional groups than can participate in side-reactions. EDC chemistry can be utilized to couple peptides to an alginate. EDC is water soluble, and provides cross-linking with no increase in the length of the cross-linking molecule, as it does not get incorporated into the bond. Other amino acids, peptides, adhesion components and/or oocyte/follicle interaction moieties or components, together with their associated starting materials, reagents and method of incorporation are known in the art. The extent to which such components are utilized is limited only by way of amount and concentration sufficient to promote adhesion or the desired cell physiological effect without adversely affecting 3-d structure of matrix formation.

Growth factors, hormones, proteins, peptides can be delivered to the follicles by incorporating these factors directly into the matrix or gel or providing them in the surrounding culture media. Direct incorporation of these growth factors, hormones, proteins and/or peptides into a bead or matrix allows one to control the initial exposure of the follicles to these factors and molecules. The presence of these factors and molecules in the culture media allows for diffusion into the bead to maintain the concentration of these factors within the bead, which may decrease due to degradation or internalization by the cells. To allow for follicle development (i.e. expansion, growth, and maturation), the bead or matrix should have the appropriate mechanical properties to permit the follicle to be encapsulated, while retaining its architecture and allowing for it to expand. The mechanics of the matrix systems of this invention can be readily controlled through the extent of crosslinking, which in turn can be controlled with matrix concentrations and the crosslinking moiety or molecule. It is preferred that the alginate beads have an alginate concentration in the range from about 0.25% (w/v) to about 2.0% (w/v). It is still more preferred that the alginate beads have an alginate concentration of between about 0.1% and 2.0%. It is still even more preferred that the alginate beads have an alginate concentration of between about 0.1% and 1.9%. It is still more preferred that the alginate beads have an alginate concentration of 0.25%, 0.5%, 0.75%, 1.0%, or 1.5% (w/v). It is still more preferred that the alginate beads have an alginate concentration of equal to, or lower than, 1.5% (w/v). The consistency of the alginate scaffolding impacts folliculogenesis and oocyte development in vitro, and the present invention maximally supports follicle growth depending on the size and stage of the follicles selected for culture.

The present invention provides for a proximal gel matrix environment at all positions around the periphery of the follicle cells, cells, or tissue. The present invention is directed to, for example, an in vitro method for maturing a preantral follicle comprising (a) suspending a preantral follicle into a non-crosslinked alginate solution, wherein the solution comprises less than 2% alginate weight per volume; (b) crosslinking the suspension, thereby forming a preantral follicle-three dimensional gel matrix; (c) culturing the preantral follicle in the three dimensional matrix, wherein the preantral follicle forms an antral cavity and whereby a cumulus-oocyte complex is formed; and (d) releasing the antral follicle from the three dimensional gel matrix. Optionally, the foregoing method may further comprise (e) culturing the released antral follicle in culture media comprising one or more pituitary hormones, wherein polar bodies are formed; and (f) releasing the oocyte from the antral follicle.

In yet another option, steps (e) and (f) in the above-described embodiment of the present invention, may be replaced with (e) isolating the cumulus-oocyte complex from the antral follicle; (f) culturing the isolated cumulus-oocyte complex in culture media comprising one or more pituitary hormones, wherein polar bodies are formed; and (g) releasing the oocyte from the cumulus-oocyte complex.

In yet another alternative to step (g) in the foregoing embodiment of the present invention, one may (g) remove the cumulus-oocyte complex from the culture.

In yet another embodiment of the present invention, an in vitro method for maturing a preantral follicle is provided, comprising (a) suspending a preantral follicle into a non-crosslinked alginate solution, wherein the solution comprises less than 2% alginate weight per volume; (b) crosslinking the suspension, thereby forming a preantral follicle-three dimensional gel matrix; (c) culturing the preantral follicle in the three dimensional matrix, wherein the preantral follicle forms an antral cavity and whereby a cumulus-oocyte complex is formed; (d) releasing the antral follicle from the three dimensional gel matrix; (e) culturing the released antral follicle in culture media comprising one or more pituitary hormones, wherein polar bodies are formed; and (f) isolating the cumulus oocyte complex from the cultured antral follicle.

Although the present invention has been described with reference to preferred embodiments, persons skilled in the art will recognize that changes may be made in form and detail without departing from the spirit and scope of the invention. For example, in many of the below-identified examples reagents such as bovine serum albumin (BSA) and αMEM are used. However, it is recognized that human serum albumin (HSA), or synthetic serum substitute (SSS), or serum protein substitute (SPS), or fetal calf serum (FCS), or polyvinyl alcohol (PVA) may be used alternative reagents to BSA. Furthermore, it is recognized that human tubal fluid (HTF), Ham's F-10 or Ham's F-12 media, G1, G2 and/or blastocyst medium, any commercially available in vitro fertilization media used for growth or maturation, KSOM media, F12-DMEM, or L-15 medium can be used as alternative media to αMEM. It is also recognized that αMEM may be combined with human chorionic gonadotropin (HCG) and/or epidermal growth factor (EGF) for maturation; and/or with collagenase and DNase I for dispersal of follicles. It is still further recognized that phosphate buffered saline (PBS) may be substituted with, for example, HEPES, MOPS, and/or any buffered media; for example, HEPES-HTF. Follicle stimulating hormone (FSH) may be human, recombinant or non-recombinant, or non-human. It is further recognized that alginate lyase, EDTA or EGTA may be used as reagent for releasing follicles from hydrogels.

### EXAMPLE 1

### Follicle Isolation, Encapsulation, and Culture to Determine Follicle Growth Regulation by ECM

C57B1/6 female mice and CBA male mice were purchased (Harlan, Indianapolis, Ind.) and maintained as a breeder colony. Protocols were approved by the IACUC at Northwestern University and animals were treated in accordance with the NIH Guide for the Care and Use of Laboratory Animals. Unless otherwise noted, all chemicals were purchased from Sigma-Aldrich (St. Louis, Mo.), stains and antibodies were purchased from Molecular Probes (Eugene, Ore.), and media formulations were purchased from Invitrogen (Carlsbad, Calif.). Sodium alginate (55-65% guluronic acid) was provided by FMC BioPolymers (Philadelphia, Pa.).

Alginate was modified with ECM molecules or RGD containing peptides. Collagen Type I isolated from rat tails (BD Biosciences, Bedford, Mass.), fibronectin from bovine plasma, and laminin and collagen Type IV purified from Engelbreth Holm Swarm Sarcoma were purchased. Aliquots of sterilized sodium alginate were reconstituted with sterile 1×PBS to a concentration of 3% (w/v), diluted to either 1.5% in PBS, or 1.5% alginate, 0.2 mg/mL ECM material, and vortexed well to mix. Alternatively, sodium alginate was covalently modified using carbodiimide chemistry to a concentration of 11.8 μmol/g alginate with GGGGRGDS peptide (CS Bio Co, San Carlos, Calif.) as previously described (18, 22) and used at a concentration of 1.5% in PBS.

Two layered secondary follicles (100-130 μm, oocyte<63 μm) and multilayered secondary follicles (150-180 μm) were mechanically isolated using insulin gauge needles in L-15 media from day 12 and day 16 C57B1/6×CBA F₁ mice, respectively. Two layered secondary follicles are type 4 or 5a and the multilayered secondary follicles are type 5b according to the classification of Pedersen and Peters. Efforts were made to maintain the follicles at 37° C. and pH 7.4 throughout the isolation and encapsulation. Follicles were then encapsulated into alginate or alginate-ECM matrices. Droplets of alginate or alginate-ECM solution (˜2-3 uL) were suspended on a polypropylene mesh (0.1 mm opening). A single follicle was pipetted into each droplet in a minimal amount of media (see FIG. 1). After all droplets had been filled, the mesh was immersed in sterile 50 mM CaCl₂ for 2 minutes to cross-link the alginate, and then rinsed in L-15 media. Individual beads were plated in 96 well plates in 100 μL of culture media composed of αMEM, 3 mg/ml BSA, 5 μg/mL insulin, 5 μg/mL transferrin, and 5 μg/mL selenium. For follicles from day 16 mice, the media was supplemented to 10 mIU/mL rhFSH (obtained through NHPP, NIDDK, and Dr. A. F. Parlow). Media used to culture multilayered secondary follicles was supplemented to 10 mIU/mL recombinant human follicle stimulating hormone (FSH).

Follicles were cultured at 37° C. in 5% CO₂ for 8 days. Every two days, half of the media volume was exchanged and follicles were examined for survival and size measurements. Follicles were designated as dead if the oocyte was no longer contained within the granulosa cells or if the granulosa cells had become dark and fragmented. Two diameters were measured for each follicle and selected images were captured. Collected media was frozen at −80° C. until assayed. 17β-estradiol and progesterone levels were determined by immunoassay (Assay Designs, Ann Arbor, Mich.). ELISA data was fit using a four point logistic equation. Intra- and inter-assay coefficients of variation were determined to be 3.1% and 8.2% for 17β-estradiol, and 4.4% and 9.1% for progesterone, respectively. Androstenedione was assayed by RIA and inhibin A by immunoassay. Intra- and inter-assay coefficients of variation were 3. 1% and 8. Intra- and inter-assay coefficients of variation were determined to be 4.9% and 11.9% for androstenedione, and 3.8% and 4.9% for inhibin A.

At the conclusion of the culture, follicles were removed from the alginate beads by degrading the gel with 10 unit/mL alginate lyase for 30 minutes at 37° C., 5% CO₂. Released follicles were then transferred to maturation media composed of αMEM, 1.5 IU/mL hCG, and 5 ng/mL EGF. Oocytes from two layered secondary follicles were mechanically denuded of granulosa cells, while oocytes from multilayered secondary follicles were maintained inside granulosa/cumulus cells. The oocytes from both size classes were incubated for an additional 14-16 hours at 37° C., 5% CO₂, and classified morphologically based on the presence or absence of a germinal vesicle and polar body. Oocytes were then fixed and processed for immunofluorescence.

### EXAMPLE 2

### Characterization of Follicle Viability and Morphology

Follicle viability one day after encapsulation was examined using a Live/Dead stain (2 μM calcein AM, 5 μM ethidium homodimer-1) and a Leica DMRXE7 confocal microscope equipped with a 40× immersion lens and Ar (488) and green HeNe (543) lasers in the Biological Imaging Facility at Northwestern University (Evanston, Ill.). An additional set of two-layered secondary follicles were encapsulated in 1.5% alginate gels and cultured for 4 days as described. The media was supplemented for the final 15 h of culture to a concentration of 1 mg/ml tetramethylrhodamine-Dextran, MW 3500. Follicles were then fixed with 3.7% formaldehyde and counterstained with 5 units/mL AlexaFluor 488 phalloidin. For comparison, a two-dimensional culture of two-layered secondary follicles was also examined, using the previously described conditions (25). Stained follicles were examined by confocal microscopy for morphology and pattern of dextran uptake.

### EXAMPLE 3

### Statistical Analysis

Follicle size and steroid levels were analyzed using a two-way ANOVA with repeated measures, or one-way ANOVA followed by Tukey-HSD for isolated time points. Categorical data was analyzed by X² analysis. All statistical calculations were done with the software package JMP 4.0.4 (SAS Institute, Cary, N.C.).

### EXAMPLE 4

### Follicle Morphology in Alginate Matrices

Two layered secondary and multilayered secondary follicles were encapsulated and cultured in alginate-based matrices. Follicles were intact after isolation and encapsulation, with a central oocyte and surrounding layers of granulosa and theca cells. Follicles were examined 24 h after isolation and encapsulation with a Live/Dead stain, and the majority of cells fluoresced green, indicating viability. The cells that appeared dead were detached from the follicle likely a result of the mechanical isolation procedure. Follicles cultured within alginate matrices maintained their spherical architecture, with a centrally placed oocyte and layers of granulosa cells. Alternatively, mouse ovarian follicles cultured on a two-dimensional substrate (for example, tissue culture plastic) had a distorted morphology with granulosa cells detaching from the follicle and migrating away from the oocyte. ECM effects on follicle development, were investigated in alginate matrices modified by physical blending with collagen I (CI), fibronectin (FN), collagen IV (CIV), and laminin (LN) and by covalent modification with RGD-containing peptides (RGD).

### EXAMPLE 5

### Characterization of Alginate-ECM Matrices

Collagen I was iodinated using the Bolton-Hunter method, and CI matrices were formed to characterize the alginate-ECM blends. Matrices formed with I¹²⁵-CI showed that the blending process results in uniform distribution of the ECM, with each bead containing a similar amount of collagen I. Although the ECM is not covalently bound, the alginate gel physically entrapped 83.5+/−1.6% of the ECM during the 8 day culture period. In addition to beads containing quantitatively similar amounts of ECM, sections stained with Sirius Red indicated that the collagen I was evenly distributed throughout the alginate matrix.

### EXAMPLE 6

### Follicle Growth Regulation by ECM

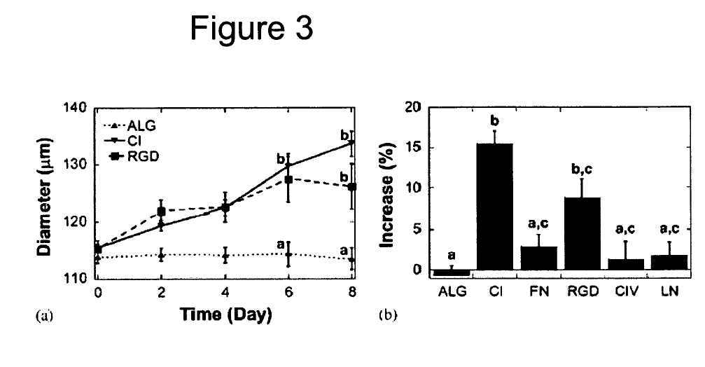

Two-layered secondary follicles (100-130 μm, oocyte<63 μm) were cultured in unmodified alginate (ALG), CI, FN, RGD, CIV, or LN matrices without follicle stimulating hormone (FSH) and their survival percentage and size compared. Survival rates ranged from 62.5% to 72.0%, with no significant difference between the different matrices (Table 3). ECM matrix significantly affected two-layered secondary follicle growth, with results dependent on ECM identity. Follicles cultured in CI and RGD grew significantly larger than follicles cultured in ALG by day 6 of culture (see FIG. 3a). At the end of the 8 day culture, follicles cultured in ALG did not increase in size (−0.6% +/−1.2%, FIG. 3b), while those cultured in CI and RGD increased significantly (15.4+/−1.6% and 8.8+/−2.3%, respectively). Follicles cultured in FN, CIV, or LN did not grow significantly larger than those cultured in ALG. Follicles cultured in these ECM-modified matrices maintained their architecture for the entire culture period, with an oocyte surrounded by layer of granulose and theca cells.

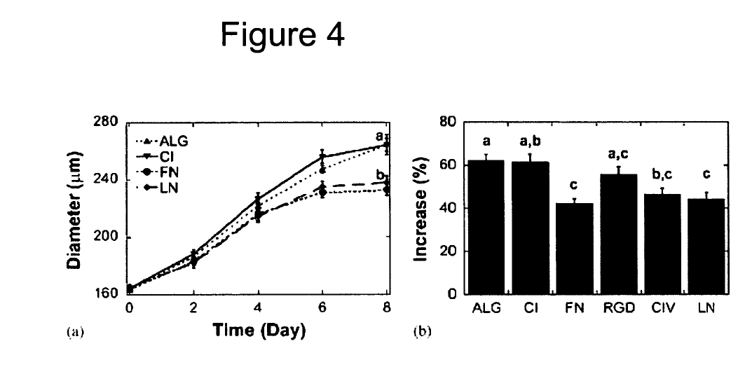

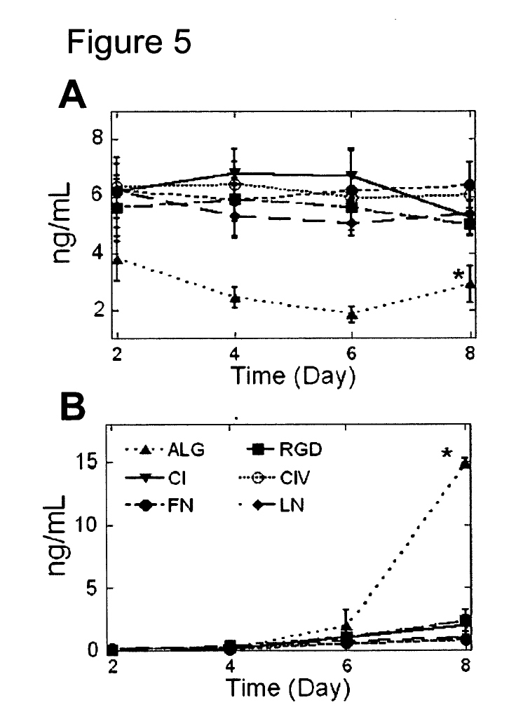

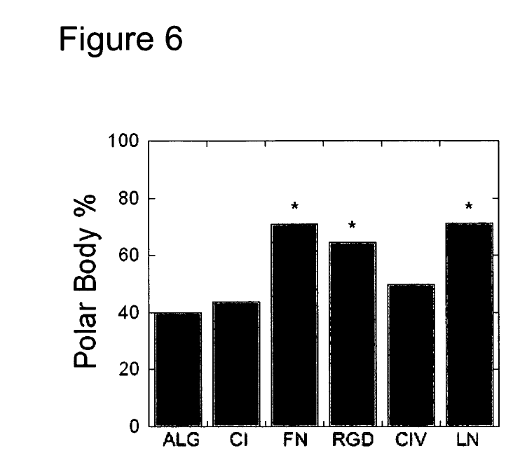

Multilayered secondary follicles (150-180 μm) were cultured in ALG, CI, FN, RGD, CIV, or LN matrices with the addition of FSH and examined for effects on survival and follicle growth. FSH was necessary for survival for this follicle stage in the various matrices examined. Follicle survival with FSH ranged from 48.1 to 71.8%, but was not significantly affected by matrix identity (Table 4). In contrast to the cultures of two-layered secondary follicles, ECM modification did not result in a significant increase in follicle growth. Rather, FN, CIV, and LN significantly decreased follicle growth compared to ALG (FIG. 4a), while CI and RGD did not significantly affect follicle growth (FIG. 4b). Although follicles did not grow as large in these modified matrices, they appeared healthy. In vivo, the granulosa cells in direct contact with the basement membrane have a lower degree of proliferation and higher degree of differentiation. Therefore, subsequent studies examined somatic cell differentiation and oocyte development.

### EXAMPLE 6

### Somatic Cell Differentiation

As follicle development progresses the somatic cells begin to perform differentiated functions, including production of steroids and inhibins. The alginate culture system provides the opportunity to directly examine whether ECM affected these processes. Progesterone and estradiol were not detected in the media collected from two-layered secondary follicle cultures, except for follicles cultured in FN, which produced low amounts of estradiol (52.1+/−5.1 μg/ml). Multilayered secondary follicles cultured in ECM modified gels secreted significantly more progesterone and significantly less estradiol than follicles cultured in ALG, p<0.05. The reduction in estradiol did not appear to result from a lack of substrate for aromatase, as androgen levels were not significantly affected by matrix composition. Additionally, inhibin A secretion was significantly higher for follicles in ALG than ECM. Estradiol levels significantly increased from day 2 to 8 for all conditions (p<0.05), while progesterone levels did not significantly increase in any of the six matrices over the culture period.

### EXAMPLE 7

### Oocyte Quality

Properly regulated follicle development is critical for the production of oocytes that are competent to resume meiosis in preparation for fertilization. The effect of ECM signaling through theca and granulosa cells on oocyte maturation was determined by characterizing the ability of the oocyte to resume meiosis. As an oocyte progresses through meiosis, the nuclear envelope (or germinal vesicle) breaks down and half of the chromosomes are physically separated from the egg into the polar body. Oocytes that have sufficiently matured will spontaneously resume meiosis when denuded of granulosa cells. ECM did not significantly affect the meiotic competency for two-layered secondary follicle cultures, with 11.5-29.4% of oocytes resuming meiosis, as evidenced by germinal vesicle breakdown (Table 3).

Although the resumption of meiosis can be examined by denuding the oocyte, in vivo it is under hormonal regulation mediated by the granulosa cells. Therefore, for multilayered secondary follicles, oocytes were examined after maturation within granulosa cells in hormone supplemented media. Culture in FN, RGD, and LN resulted in a significant increase in rate of polar body formation compared to ALG (Table 3). Oocytes were further examined to characterize the quality of the meiotic spindle by staining for tubulin and chromatin. These experiments revealed that cultured oocytes were undergoing the normal stages of meiotic progression. Oocytes from ALG, CI, and CIV were primarily observed in prometaphase I with the chromatin condensed and tubulin forming a spindle (or in metaphase I, with a characteristic barrel shaped spindle). Oocytes from FN, RGD, and LN had a more compact metaphase II spindle and a polar body. Importantly, both metaphase I and II spindles had chromosomes aligned at the equator of the spindle, an indicator that normal chromosome division, necessary to avoid aneuploidy, is occurring. No significant differences in the percentage of aligned spindles were measured between matrix conditions.

### EXAMPLE 8

### Follicle Isolation, Encapsulation, and Culture to Determine FSH Regulation of Two-Layered Secondary Follicles

C57BL/6 female mice and CBA male mice were purchased (Harlan, Indianapolis, Ind.) and maintained as a breeder colony at Northwestern University (Evanston, Ill.). Animals were housed in a temperature and light controlled environment on a 12L: 12D photoperiod and provided with food and water ad libitum. Chow provided was Harlan Teklad Global irradiated 2919 which does not contain soybean or alfalfa meal and therefore contains minimal phytoestrogens. Animals were treated in accordance with the NIH Guide for the Care and Use of Laboratory Animals and the established IACUC protocol at Northwestern University. Sodium alginate (55-65% guluronic acid) was provided by FMC Biopolymers (Philadelphia, Pa.).

Two-layer secondary follicles (100-130 μm, oocyte 53-63 μm) and multilayer secondary follicles (150-180 μm, oocyte 61-74 μm) were mechanically isolated using insulin gauge needles in L-15 media from day 12 and day 16 C57BL/6×CBA F1 mice, respectively. Two-layer secondary follicles are type 4 or 5a and multilayer secondary follicles are type 5b according to the classification of Pedersen and Peters. Efforts were made to maintain the follicles at 37° C. and pH 7 throughout the isolation and encapsulation. Two-layer secondary follicles were encapsulated into sterile alginate-collagen I matrices composed of 1.5% (w/v) alginate and 0.2 mg/mL collagen I (BD Biosciences, Bedford, Mass.) and multilayer secondary follicles were encapsulated into sterile alginate matrices composed of 1.5% (w/v) alginate, as these matrix formulations promoted the maximum follicle growth (unpublished observations). Droplets of alginate or alginate-ECM solution (2-3 μl) were suspended on a polypropylene mesh (0.1 mm opening, McMaster-Carr, Atlanta, Ga.). A single follicle was pipetted into each droplet in a minimal amount of media. After all droplets had been filled, the mesh was immersed in sterile 50 mM CaCl₂ for 2 minutes to cross-link the alginate, and then rinsed in L-15 media. Beads were plated (one follicle per well) in 96 well plates in 100 uL of culture media composed of αMEM, 3 mg/mL BSA, 5 μg/mL insulin, 5 g/mL transferrin, and 5 ng/mL selenium, without androgen supplementation. Media were supplemented with FSH to final concentrations from 0 to 50 mIU/mL with recombinant human FSH (obtained through NHPP, NIDDK, and Dr. A. F. Parlow). Follicles were cultured at 37° C. in 5% CO₂ for 8 days. Every two days, half of the media volume was exchanged and follicles were examined for survival and size measurements, using an inverted Leica DM IRB microscope with transmitted light and phase objectives (Leica, Bannockburn, Ill.). Follicles were designated as dead if the oocyte was no longer contained within the granulosa cells or if the granulosa cells had become dark and fragmented. Two diameters were measured for each follicle and collected media were frozen at −80° C. until assayed.

17β-estradiol and progesterone levels were determined by immunoassay (Assay Designs, Ann Arbor, Mich.). ELISA data were fit using a four point logistic equation. Intra- and inter-assay coefficients of variation were determined to be 3.1% and 8.2% for 17β-estradiol, and 4.4% and 9.1% for progesterone, respectively. The sensitivity limit for 17β-estradiol was 30 pg/mL and the sensitivity limit for progesterone was 62.5 pg/mL. Collected media were also analyzed on an YSI 2700 Select Biochemistry Analyzer for L-lactate and glucose levels.

At the conclusion of the culture, follicles were removed from the alginate beads by degrading the gel with 10 units/ml alginate lyase for 30 minutes at 37° C., 5% CO₂. Released follicles were then transferred to maturation media composed of αMEM, 1.5 IU/mL hCG, and 5 ng/mL EGF. After an incubation of 14-16 hours at 37° C., 5% CO₂, oocytes were classified morphologically based on the presence or absence of a germinal vesicle and polar body. Oocytes were classified as degenerated if the cytoplasm was fragmented or shrunken from the zona pellucida. Oocytes were then fixed and processed for irnmunofluorescence. Oocytes were stained with a 1:400 dilution of monoclonal anti-o-tubulin (Sigma), detected with a 1:500 dilution of AlexaFluor 488 Goat Anti-Mouse (Molecular Probes, Eugene, Ore.), and mounted in VectaShield with DAPI (Vector Laboratories, Burlingame, Calif.) to examine the meiotic spindles. For control in vitro maturation oocytes for two-layer secondary follicle cultures, day 18 mice were primed with 5 IU of PMSG, and then denuded oocytes were collected from large follicles on day 20. For control in vitro maturation oocytes for multilayer secondary follicle cultures, day 22 mice were primed with 5 ID of PMSG and then cumulus-oocyte complexes were collected on day 24. Control in vivo matured oocytes for multilayer secondary follicle cultures were obtained from ovulated cumulus oocyte complexes from day 24 mice primed with 5 IU of PMSG for 48 hours and 5 ID of hCG for 14 hours prior to collection.

Follicles cultured in alginate beads were fixed with 4% paraformaldehyde for 1 hour at the completion of the culture period, dehydrated through an ethanol series, and then embedded in LR White (Electron Microscopy Sciences, Hatfield, Pa.). The embedded beads were then sectioned as 1 pm sections (Cell Imaging Facility, Northwestern University, Chicago, Ill.) and stained with hematoxylin for 5 minutes to examine granulosa cell morphology.

Follicles were cultured as described above for the first 2 days of culture. Media was then exchanged and replaced with media supplemented with 0.2 μpCi [methyl-³H] thymidine per follicle (Amersham Biosciences, Piscataway, N.J.). After 24 hours, 5 beads were collected for each replicate, washed twice with 1×PBS, and then dissolved in 10 mM EDTA. ³H thymidine incorporation was then assayed as has been previously described in the art. Non-specific incorporation was determined using empty alginate gels For two-layer secondary follicle cultures, two or three independent cultures of 3050 follicles each were performed for each FSH dose. For multilayer secondary follicles, two to four independent cultures of 10-30 follicles each were performed for each FSH dose. Follicle size, steroid, and lactate data were analyzed using a two-way ANOVA with repeated measures, or one-way ANOVA followed by Tukey-HSD for isolated time points with a Bonferroni correction for multiple comparisons. Categorical data was analyzed by X2 analysis. A p-value of less than 0.05 was considered statistically significant. All statistical calculations were done with the software package JMP 4.0.4 (SAS Institute, Cary, N.C.).

### EXAMPLE 10

### FSH Regulation of Two-Layer Secondary Follicles

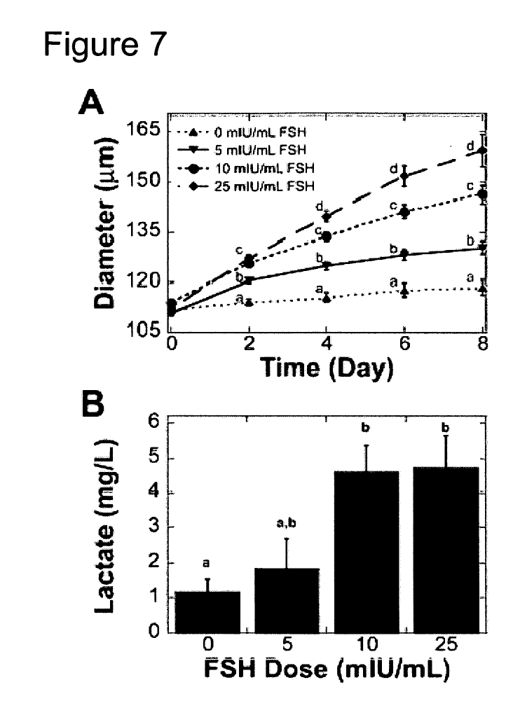

Two-layer secondary follicles (Type 4 or 5a, 100-130 μm) were cultured in alginate-collagen I gels with 0, 5, 10, or 25 mIU/mL recombinant human follicle stimulating hormone (FSH) for 8 days. Collagen-I alginate matrices promoted growth of two-layer secondary follicles in the absence of FSH. Survival of two-layer secondary follicles was not significantly affected by FSH dose, but two-layer secondary follicles grew significantly larger with FSH treatment (Table 1). Follicles were designated as dead if the oocyte was no longer contained within the granulosa cells or if the granulosa cells had become dark and fragmented. The effect of FSH on two-layer secondary follicle growth was apparent by the second day of culture, with follicles cultured in 10 and 25 mIU/mL FSH significantly larger than those cultured with 0 or 5 mIU/mL FSH (FIG. 7A). Increased dosages of FSH also resulted in a significant increase in the accumulation of lactate in the media at the end of culture (FIG. 7B). Lactate production did not correspond linearly with follicle size, indicating that granulosa cells of two-layer secondary follicles cultured with higher doses of FSH had an increased metabolism.

Progesterone and estradiol were not detected at any time for two-layer secondary follicles cultured without FSH. Progesterone levels increased significantly between day 2 and day 8 of the culture for follicles cultured with 10 or 25 mIU/ml FSH, but there was not significant difference between FSH doses at the individual time points. Estradiol levels were also significantly higher at the end of the culture period, even though culture media were not supplemented with exogenous androgen. Additionally, culture with either 10 or 25 mIU/ml FSH resulted in significantly higher estradiol levels on day 8 of culture compared to culture with 5 mIU/ml FSH. FSH dose did not significantly affect the percentage of oocytes that were competent to resume meiosis at the conclusion of culture. The majority of the oocytes examined were arrested at prophase I with an intact germinal vesicle. Oocytes that had resume meiosis were arrested in metaphase I.

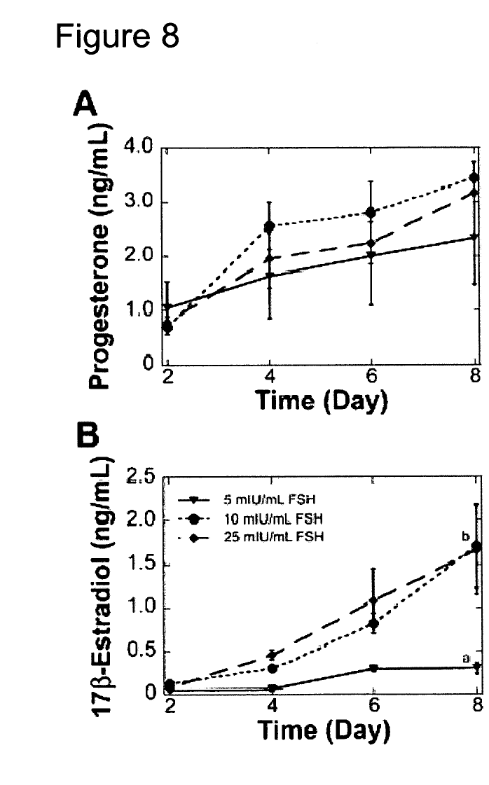

In this system, two-layered secondary follicles cultured in alginate-collagen I gels were FSH responsive, with increased follicle growth and lactate production (FIG. 7), and increased estradiol secretion relative to follicles cultured without FSH (FIG. 8). An increase in lactate production has previously been shown to coincide with rapid growth and the onset of estradiol secretion in cultured intact follicles, indicating that the two-layered secondary follicles cultured in alginate-collagen I gels differentiated in response to the increased doses of FSH. This result was in contrast to previous studies of two-layered secondary follicles isolated from immature mice and cultured in serum-free conditions, which did not respond to FSH without additional supplementation with activin or treatment with diethylstilbestrol. FSH has been shown to promote growth and differentiation for serum-supplemented culture of two-layered secondary follicles on two-dimensional substrates in a manner similar to the results of these serum-free studies in alginate-collagen I gels (FIGS. 7 and 8); without FSH, estradiol and progesterone were not detected and little granulosa cell proliferation occurred. However, FSH was not necessary for survival of two-layered secondary follicles in the alginate-collagen I matrices (Table 1) and limited growth occurred without FSH (FIG. 7), indicating that this stage of follicle was not FSH dependent in this system. In contrast, follicles of the same size class cultured on two-dimensional substrates with serum required FSH for survival and development. See Cortvrindt and Van Steirteghem, Hum. Reprod. 1997; 12:75-768. Thus, this three-dimensional alginate culture system provided a more in vivo like dynamic for follicle progression.

The oocytes form two-layered secondary follicles in the alginate cultures were immature in comparison to age-matched in vitro matured controls (Table 2). The apparent slower development of the oocytes cultured in vitro has been reported previously for two-dimensional culture systems. See Eppig et al., Biol. Reprod. 1996; 54:197-207.

### EXAMPLE 11

### FSH Regulation of Multilayered Secondary Follicles

Multilayered secondary follicles (Type 5b, 150-180 μm) were cultured in alginate hydrogels for 8 days with 0, 5, 10, 25, or 50 mIU/mL FSH. Multilayered secondary follicle survival was significantly affected by FSH dose, with a maximum survival of 72.0% and 69.2% at 5 and 10 mIU/ml, respectively (see Table 1). Sections of follicles cultured with 0, 10, or 50 mIU/mL FSH were examined to better characterize the health of the granulosa cells. Culture without FSH resulted in a large number of pyknotic nuclei throughout the follicle. This morphology was not observed in sections of follicles cultured with 10 mIU/mL FSH. With the further increase in the dose to 50 mIU/mL FSH, a large number of pyknotic nuclei were again observed. Unlike the follicles cultured without FSH, however, pyknotic cells from FSH treated cultures were found primarily around the oocyte rather than the periphery of the follicle.

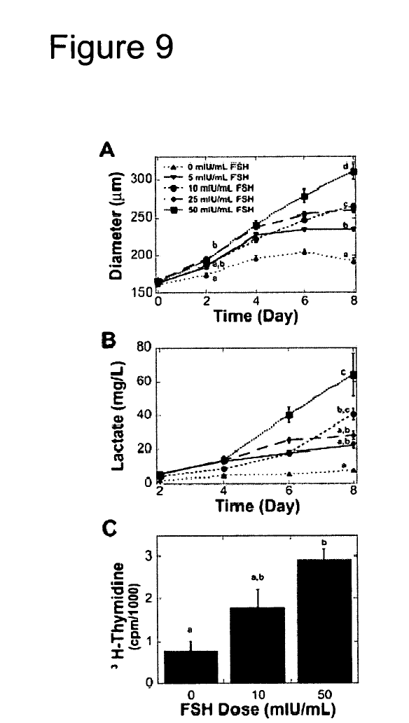

Multilayer secondary follicle growth was dependent on FSH dose (Table 1). The difference in follicle size was first detected on day 2 of the culture, with follicles cultured with 25 and 50 mIU/mL significantly larger than those cultured without FSH (FIG. 9A). At the completion of the culture period, follicle size showed a dose dependent response. In addition, multilayer secondary follicles had an increased production of lactate with increased doses of FSH (FIG. 9B). A corresponding decrease in glucose was observed in the conditioned media (data not shown). Somatic cell proliferation was also assessed using a ³H-thymidine incorporation assay for culture with 0, 10 and 50 mIU/mL FSH. For the day 2 to day 3 period of culture, follicles in all media conditions incorporated ³H-thymidine, indicating that DNA replication and therefore cellular proliferation had occurred. Follicles cultured with 50 mIU/mL FSH incorporated significantly more ³H-thymidine compared to follicles cultured without FSH (FIG. 9C), which was in agreement with the observed increase in follicle size on day 2 for follicles treated with 50 mIU/mL FSH (FIG. 9A).

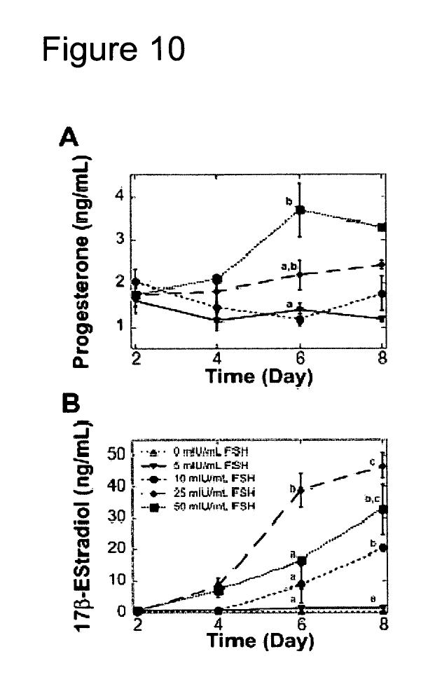

Progesterone and estradiol secretion by multilayer secondary follicles was regulated by FSH in a dose-dependent manner. Progesterone was not detected from follicles cultured without FSH (data not shown), but was significantly increased on day 6 from cultures with 50 mIU/mL FSH relative to cultures with 5 or 10 mIU/mL FSH (FIG. 10A). However, this difference was no longer significant on day 8 of culture. Estradiol levels were also dependent on FSH dose with levels significantly higher at the conclusion of the culture for all FSH doses, but not for follicles cultured without FSH. FSH induced a dose dependent increase in estradiol secretion for multilayer secondary follicles cultured with 5, 10, and 25 mIU/mL FSH (FIG. 10B). Further increases in the FSH dose to 50 mIU/mL resulted in a small, but not significant, decrease in estradiol compared to 25 mIU/mL FSH.

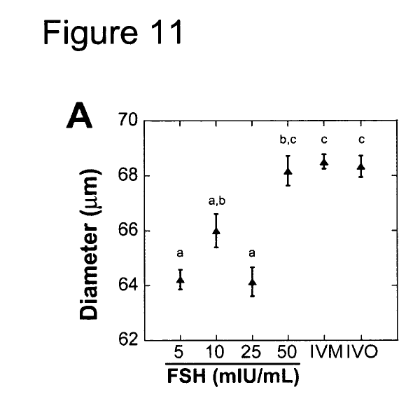

Oocyte meiotic competence was affected by FSH dose as well, with 84.6% of oocytes from cultures without FSH appearing degenerated, which was significantly higher than any FSH treated culture (Table 2). Follicles cultured with 5 mIU/mL of FSH had the highest rate of progression to metaphase II, as evidenced by a polar body (Table 2). However, oocytes cultured with 50 mIU/mL FSH were the largest in size and were not significantly different than in vitro and in vivo matured controls (FIG. 11A, p>0.05). Oocyte metaphase II spindles were a characteristic barrel shape with chromatin aligned at the spindle equator. There was no significant difference in the percentage of aligned spindles among the FSH treatments (data not shown).

Multilayered secondary follicle growth in the alginate matrix was a result of granulosa cell proliferation, the rate of which depended on FSH dose (FIG. 9). Multilayered secondary follicles grew without FSH, but follicle growth slowed after day 2, and follicles actually decreased slightly in size from day 6 to day 8 (FIG. 9). A similar trend was seen for early antral follicles cultured without FSH on a two-dimensional substrate. See, for example, Spears et al., J. Reprod. Fertil. 1998; 113:19-26; and Nayudu and Osborn, J. Reprod. Fertil., 1992; 95: 349-362. Follicles grew significantly larger with increased doses of FSH, indicating that the three-dimensional support of the alginate matrix did not restrict follicle growth. The increased rate of granulosa cell proliferation may have uncoupled granulosa cell-granulosa cell interactions or granulosa cell-oocyte communication, affecting follicle survival. Alternatively, the continual exposure to FSH may have led to FSH receptor desensitization, resulting in lowered follicle survival and flattened follicle growth curves, as was observed for multilayered secondary follicles cultured with 5 or 25 mIU/mL FSH.

Granulosa cells from mature follicles secrete large amounts of steroids, particularly in response to gonadotropin signaling as the dominant follicle matures. In the present alginate system, granulosa cells secreted progesterone in response to increased FSH, while when cultured in the absence of FSH progesterone was not detected (FIG. 10). There was a significant increase on day 6 for cultures treated with 50 mIU/mL FSH, indicating a possible premature luteinization of the granulosa cells. The production of estradiol by the cultured multilayered secondary follicles indicated a functioning theca, as the cultures were not supplemented with androgen. Additionally, FSH regulated estradiol secretion, as expected from the 2 cell-2 gonadotropin model and cultures of multilayered secondary follicles on two-dimensional substrates. Unlike follicle growth and lactate production, this was not a strictly dose dependent response, as the maximum amount of estradiol was achieved with 25 mIU/mL FSH and not 50 mIU/mL FSH (FIG. 10). This result may correspond to follicle growth, as 25 mIU/mL FSH did not significantly increase follicle growth relative to 10 or 50 mIU/mL FSH. Therefore, granulosa cells at this dose of FSH may be proliferating less, resulting in increased differentiation.

Multilayered secondary follicles also produced oocytes that were competent to resume meiosis and progress to metaphase II, an important functional endpoint of the culture system. Oocytes from follicles cultured without FSH were not healthy, appearing dark with a fragmented cytoplasm. The poor morphology of oocytes from cultures without FSH was not unexpected, based on the reduced follicle survival (Table 1) and extensive granulosa cell apoptosis seen in these follicles, and previous reports of poor oocyte quality from follicles cultured without FSH. Culture with even the lowest dose of FSH significantly improved oocyte health compared to no FSH. It has been shown that in vivo treatment with FSH induced withdrawal of transzonal projections, which corresponded to changes in oocyte transcriptional activity and increased rates of oocyte meiotic competence.

### EXAMPLE 12

### Follicle Isolation, Encapsulation and Analysis of Alginate Hydrogel Cultures

Immature follicles were isolated from prepubertal, 12-day-old female F1 hybrids (C57BL/6j×CBA/Ca), and sperm was prepared from proven CD1 male breeders. Animals were housed in a temperature- and light-controlled environment (12 h light: 12 h dark) and provided with food and water ad libitum. Animals were fed Teklad Global irradiated 2919 chow, which does not contain soybean or alfalfa meal and therefore contains minimal phytoestrogens. Animals were treated in accordance with the NIH Guide for the Care and Use of Laboratory Animals and the established IACUC protocol at Northwestern University.

Sodium alginate (55-65% glucuronic acid) was provided by FMC BioPolymers (Philadelphia, Pa.). Alginate was dissolved in deionized water to a concentration of 1% (w/v) and then purified with activated charcoal (0.5 g charcoal/g alginate) to remove organic impurities and improve the purity of the alginate. Following charcoal treatment, alginate solution was sterile filtered through 0.22 μm filters, lyophilized within Steriflip conical tubes (Millipore, Billerica, Mass.) and sterilely aliquoted. Aliquots of charcoal-stripped and sterilized sodium alginate were reconstituted with sterile 1×PBS to concentrations of 1.5%, 1.0%, 0.5% and 0.25% (w/v) for each experiment.

Two layered secondary follicle (100-130 μm, type 4) were isolated from 12-day-old female mice and encapsulated into alginate beads prepared at various concentrations (1.5%, 1.0%, 0.5% and 0.25%) (w/v) as described previously with slight modifications. Follicles were mechanically isolated using insulin gauge needles in L15 media (Invitrogen, Carlsbad, Calif.) containing 1% FCS. Individual follicles were maintained in αMEM/1% FCS at 37° C., 5% CO₂ for 2 hours before encapsulation. Only those follicles displaying the following characteristics during the 2-hour pre-incubation period were selected for encapsulation and culture: 1) diameter of 100-130 μm; 2) intact with some attached, fibroblast-like theca cells; 3) a visible, immature oocyte that was round and centrally located within the follicle.

Single follicles were pipetted into the middle of each alginate droplet (2-3 μl) suspended on a polypropylene mesh (0.1 mm opening, McMaster-Carr, Atlanta, Ga.). When encapsulating follicle into the 1.5% and 1.0% alginate beads, the mesh was immediately immersed in sterile encapsulation solution (50 mM CaCl2, 140 mM NaCl). When encapsulating follicles into the 0.5% and 0.25% alginate beads, the mesh was turned over after follicle placement, and then flipped into the encapsulation solution by shaking the mesh very quickly. Alginate beads were left in the encapsulation solution for 2 minutes to cross-link the alginate, and then rinsed in culture media (αMEM with 10 mIU/ml rFSH, 3 mg/ml BSA, 1 mg/ml bovine fetuin, 5 μg/ml insulin, 5 μg/ml transferrin, and 5 ng/ml selenium). Alginate beads containing a single follicle were plated one follicle per well in 96-well plates in 100 μl of culture media. Fetuin, dialyzed extensively against embryo culture-grade water and lyophilized, was added to prevent zona pellucida (ZP) hardening. Throughout isolation, encapsulation and plating, follicles were maintained at 37° C. and pH7.

Encapsulated follicles were cultured at 37° C. in 5% CO₂ for either 8 days (for RNA extraction and oocyte size measurement) or 12 days (for IVMIIVF experiment and oocyte size measurement). Every other day, half of the media (50 ml) was exchanged and stored at −80° C. Follicle survival and diameter were assessed using an inverted Leica DM IRB microscope with transmitted light and phase objectives (Leica, Bannockburn, Ill.). Follicles were designated dead if the oocyte was no longer surrounded by a granulosa cell layer or if the granulosa cells had become dark and fragmented and the follicle had decreased in size. After 8 or 12 days culture, the culture media was replaced by 100 μl L15 medium containing 10 units/ml alginate lyase (Sigma-Aldrich) for 30 minutes at 37° C. Follicles were removed from the degraded alginate bead and all remaining alginate was removed in a separate IVF dish containing L15 medium with 1% FCS.

### EXAMPLE 13

### Follicle and Oocyte Measurement

The diameters of oocytes from in vitro-cultured follicles were obtained on days 8 and 12. The diameter of follicles containing oocytes was measured in duplicate from the outer layer of theca cells using Image J 1.33U and based on a calibrated ocular micrometer. Immature oocytes were denuded by gentle aspiration through glass pipettes. The oocyte diameter was measured without the ZP.

### EXAMPLE 14

### Oocyte Maturation, Fertilization and Embryo Culture

After 12 days of culture, follicles were retrieved from the alginate bead and transferred to maturation media composed of αMEM, 10% FCS, 1.5 IU/ml hCG and 5 ng/ml EGF for 16 hours at 37° C., 5% CO₂. Oocytes were then denuded from the surrounding cumulus cells by treatment with 0.3% hyaluronidase and gentle aspiration through a polished drawn glass pipette. The oocytes were considered to have undergone germinal vesicle breakdown (GVBD) if a germinal vesicle was not visible. If a polar body was present in the perivitelline space, the oocytes were classified as metaphase II (MII). Fragmented or shrunken oocytes were classified as degenerated (DG).

Motile sperm was prepared from a sperm suspension collected from the cauda epididymis of proven CD1 male breeder mice using Percoll gradient-centrifugation (PGC). PGC sperm was capacitated in IVF medium (KSOM [Specialty Media, Phillipsburg, N.J.] supplemented with 3 mg/ml BSA, 5.36 mM D-Glucose) for 30 minutes. Fifteen to 20 MII oocytes were placed in 50 μl IVF medium microdrops containing 1×10⁶ sperm/ml and incubated under mineral oil for 7-8 hours at 37° C., 5% CO₂. Oocytes were then washed three times in fresh KSOM to remove all bound sperm and transferred into a 20 μl fresh KSOM drop overnight. Embryos that cleaved to the 2-cell stage were characterized as fertilized. Embryos were washed in KSOM and cultured until the blastocyst stage. The blastocyst formation rate was scored at day 5 of culture.

Methods for producing mammalian embryonic stem cells are based on the findings of the present invention that a blastocyst can be obtained from a fertilized oocyte in a surprisingly high probability (30% blastocyst formation from fertilized oocytes matured using the in vitro methods described herein). This high rate of blastocyst formation is the result of the herein described methods for maturing preantral follicles and obtaining the resultant mature oocytes. For example, one such method is directed to the in vitro production of a stem cell line comprising the steps of: (a) suspending a preantral follicle into a non-crosslinked alginate solution, wherein the solution comprises less than 2% alginate weight per volume; (b) crosslinking the suspension, thereby forming a preantral follicle-three dimensional gel matrix; (c) culturing the preantral follicle-three dimensional gel matrix in culture containing one or more pituitary hormones (for example, a follicle stimulating hormone), wherein the preantral follicle forms an antral cavity and whereby a cumulus-oocyte complex is formed; (d) releasing the antral follicle from the three dimensional gel matrix; (e) culturing the released antral follicle in culture media comprising one or more pituitary hormones (for example, human chorionic gonadotropin and luteinizing hormone), wherein polar bodies are formed; (f) releasing the oocyte from the antral follicle; (g) fertilizing the oocyte in vitro, thereby forming a pre-implanted embryo; (h) culturing the resultant embryo in vitro, wherein a blastocyst is formed; and (i) deriving stem cells derived from the blastocyst. The preantral follicle may be, for example, a two-layered secondary follicle or a multilayer secondary follicle.

In another example, a stem cell line is produced by a method comprising the steps of: (a) suspending a preantral follicle into a non-crosslinked alginate solution, wherein the solution comprises less than 2% alginate weight per volume; (b) crosslinking the suspension, thereby forming a preantral follicle-three dimensional gel matrix; (c) culturing the preantral follicle-three dimensional gel matrix in culture containing one or more pituitary hormones (for example a follicle stimulating hormone) for about 8 hours; (d) releasing the follicle from the three dimensional gel matrix; (e) culturing the released follicle in culture media comprising one or more pituitary hormones (for example, human chorionic gonadotropin or luteinizing hormone), wherein polar bodies are formed; (f) releasing the oocyte from the follicle; (g) fertilizing the oocyte in vitro, thereby forming a preimplantation embryo; (h) culturing the resultant embryo in vitro, wherein a blastocyst is formed; and (i) deriving stem cells derived from the blastocyst.

In yet another method, a stem cell line may be produced using a method comprising the steps of: (a) suspending a preantral follicle into a non-crosslinked alginate solution, wherein the solution comprises less than 2% alginate weight per volume; (b) crosslinking the suspension, thereby forming a preantral follicle-three dimensional gel matrix; (c) culturing the preantral follicle-three dimensional gel matrix in culture containing one or more pituitary hormones (for example, follicle stimulating hormone), wherein a mature follicle is formed; (d) releasing the mature follicle from the three dimensional gel matrix; (e) culturing the released mature follicle in culture media comprising one or more pituitary hormones (for example, luteinizing hormone and/or human chorionic gonadotropin), wherein polar bodies are formed; (f) releasing the oocyte from the mature follicle; (g) fertilizing the oocyte in vitro, thereby forming a preimplantation embryo; (h) culturing the resultant embryo in vitro, wherein a blastocyst is formed; and (i) deriving stem cells derived from the blastocyst.

### EXAMPLE 15

### Characterization of Follicle Functionality

After 8 days of culture, follicles were isolated from the alginate beads as described above. Immature denuded oocytes were separated from the surrounding somatic cells by gentle aspiration through glass pipettes in L15 media. Oocytes and somatic cells were separately transferred into two clean tubes with a minimal amount of media. Total RNA was purified from both oocytes and somatic cells by using Stratagene Absolutely RNA Microprep Kit (Cedar Creek, Tex.) according to the manufacturer's procedure. Total RNA was reverse transcribed into first-strand cDNA (Invitrogen, SuperScript First-Strand Kit) using random hexamer primers and stored at −20° C. Real time PCR was used to compare the expression levels of FSH-receptor (FSHR), LH-receptor (LHR) and Connexin 43 (Cx43) levels in somatic cells and Growth Differentiation Factor 9 (GDF9) and Maternal Antigen that Embryos Require (MATER) in denuded oocytes. GAPDH was used for endogenous control. All real-time PCR experiments were performed using Taqman probes. RT reactions run in the absence of reverse transcriptase served as a negative control.

### EXAMPLE 16

### Hormone Assays

Androstenedione, 17β-estradiol and progesterone were measured in conditioned media collected on follicle culture days 4, 6, 8, 10, and 12 using commercially available radioimmunoassay kits. Media collected from wells containing no follicles was used as the assay control.

### EXAMPLE 17

### Statistical Analysis

Follicle size, survival rate, antral and theca growth rate, steroid production, and IVF and embryo culture were conducted using four independent cultures. Three independent cultures were used for measurement of denuded oocyte size and RNA preparation. Data were analyzed using a one-way ANOVA followed by a paired t-test. A p-value of less than 0.05 was considered statistically significant.

### EXAMPLE 18

### Evaluation of in vitro Cultured Follicle Growth

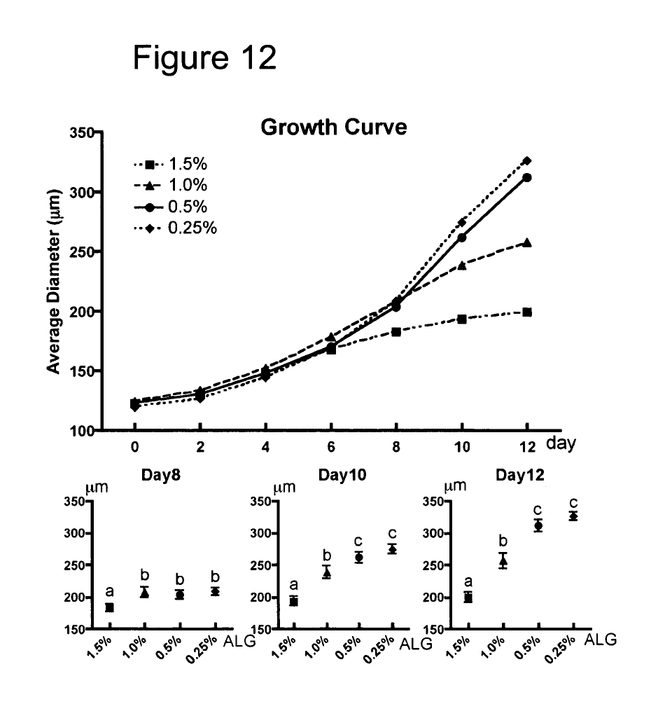

Follicles maintained their three-dimensional structures in all alginate bead concentrations tested. Survival rates did not differ significantly among the different groups. During the first 6 days of culture, follicle sizes among the four groups were not significantly different; however, after 8 days of culture, follicle growth was negatively correlated with alginate concentration (FIG. 12 and Table 5). During the last 4 days of culture, follicles embedded in 0.5% and 0.25% alginate had linear growth curves and were significantly larger than follicles grown in 1.5% and 1.0% alginate. Very few antral follicles developed in the 1.5% alginate cultures, whereas 73.2% and 86.2% of follicles grown in 0.5% and 0.25% alginate, respectively, developed an antrum (Table 5). In addition, multiple laminar-like theca cell layers were observed after day 8 among most of the follicles cultured in 0.5% and 0.25% alginate.

### EXAMPLE 19

### Steroid Production In Vitro

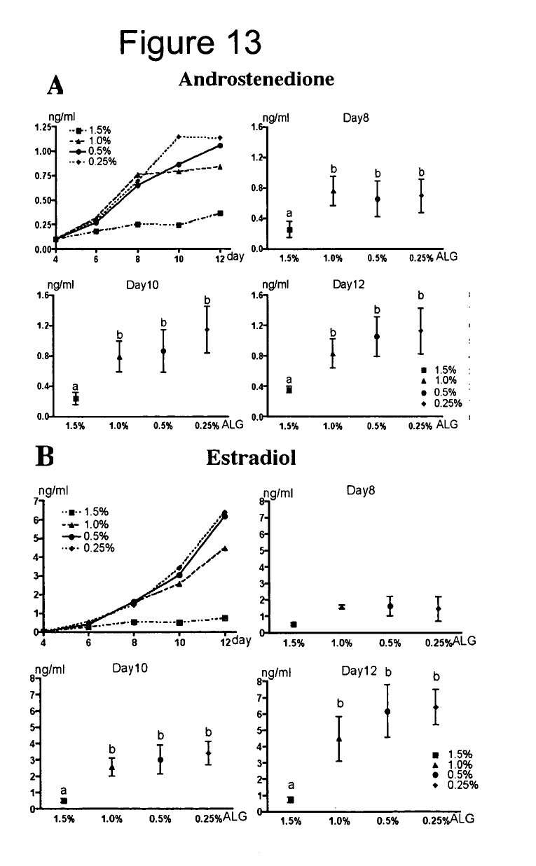

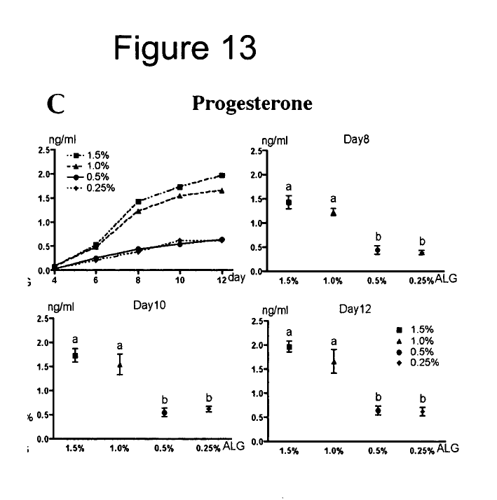

Secretion patterns of androstenedione, 17β-estradiol and progesterone from each group of in vitro-cultured follicles were consistent with the observed changes in follicle morphology and differentiation (FIG. 13). All steroid levels increased significantly from baseline over time. At day 8, follicles grown in lower alginate concentrations showed significant increases in androstenedione (FIG. 13A) and estradiol (FIG. 13B) secretion, though estradiol levels rose more slowly than androstenedione over the culture period. Significant increases in estradiol production by follicles grown in lower concentration alginate were not observed until day 10 and 12. In contrast, progesterone secretion was significantly lower in follicles encapsulated in 0.5% and 0.25% alginate compared with those grown in 1.5% and 1.0% alginate (FIG. 13C).

### EXAMPLE 20

### Characterization of Differential Gene Expression by Real-time PCR

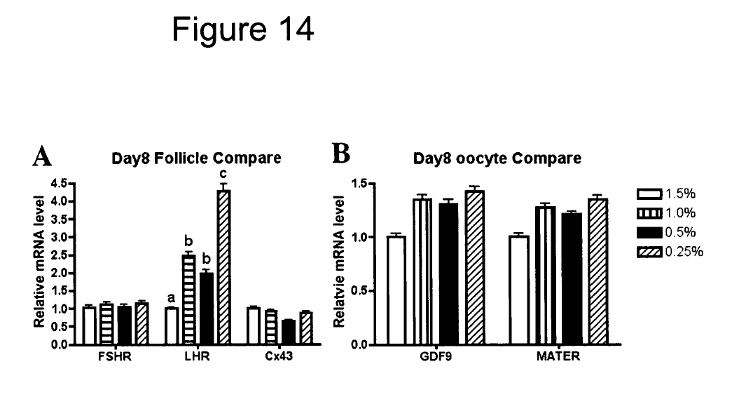

The differential expression levels of three genes (FSHR, LHR and Cx43) in each alginate group at day 8 of culture were compared using real-time PCR. Day 8 cultured follicles were selected for these experiments because growth and morphology differences among the test groups developed by this time point (FIG. 12). There was no significant difference in FSHR and Cx43 expression in the different alginate groups (FIG. 14A). LHR expression was significantly up-regulated in follicles at lower alginate concentrations, with follicles encapsulated in 0.25% alginate having approximately 4 times higher LHR expression than those grown in 1.5% alginate (FIG. 14A).

### EXAMPLE 21

### Oogenesis Oocyte Growth

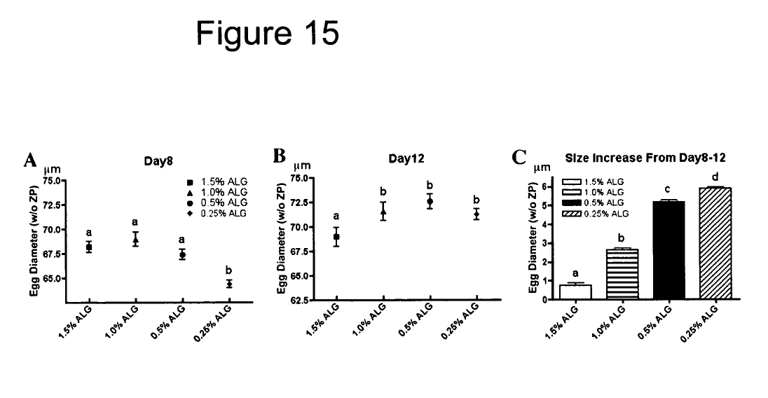

To compare the oocyte size from different alginate concentration groups, GV 215 oocytes were denuded by gentle aspiration through glass pipettes after 8 and 12 days of culture. On day 8, the average diameter of oocytes cultured in 0.25% alginate was smaller than those cultured in the other concentration alginates (FIG. 15A). However, by day 12, the average diameter of oocytes cultured in the lower concentrations of alginate had increased while oocyte size in 1.5% alginate remained unchanged (FIG. 15B). The 220 overall increase in oocyte size between day 8 and day 12 was highest in follicles grown in the lower alginate concentrations (FIG. 15C).

### EXAMPLE 22

### Meiotic Competence. IVF and Embryo Development

After 12 days of culture, follicles were separated from alginate beads and stimulated with hCG and EOF for 16 hours. Mucification was observed for all follicles if they had formed an antrum by the end of culture (data not shown). No significant differences of GVBD rates were found among the alginate concentration groups (Table 6). However, more oocytes cultured in 0.5% and 0.25% alginate extruded the first polar body compared with those cultured in 1.5% and 1.0% alginate (Table 6).

Subsequent IVF of mature oocytes resulted in 2-cell embryos after 24 hours. The fertilization rates of oocytes cultured in 0.25% alginate were significantly higher than those cultured in 0.5%, 1.0% and 1.5% alginate. After 5 days, 29.4% embryos from the 0.25% alginate group developed to expanded blastocysts, whereas no blastocysts developed from embryos from the other alginate concentration groups (Table 6).

### EXAMPLE 23

### Characterization of Differential Gene Expression by RT-PCR

The differential expression levels of two oocyte specific genes (GDF9 and MATER) in oocytes grown in each alginate concentration group were compared by real-240 time PCR. In order to eliminate the influence of somatic cells, denuded oocytes were used for total RNA extraction and PCR amplification. Although GDF9 and MATER expression were slightly lower in the 1.5% alginate group compared with the other groups, the difference was not a statistically significant (FIG. 14B).

### EXAMPLE 24

### Tissue-Engineered Follicles Produce Live, Fertile Offspring

Immature follicles were isolated from prepubertal, 16-day-old female F 1 hybrids (C57BL/6j×CBA/Ca), and sperm was prepared from proven CD1 male breeders. Eight-to 10-week-old CD1 female mice that had been mated to vasectomized CD1 male mice served as pseudopregnant mice for IVF. Animals were housed in a temperature- and light-controlled environment (12 h of light: 12 h of dark) and provided with food and water ad libitum. Animals were fed Teklad Global (Madison, Wis.) irradiated 2919 chow, which does not contain soybean or alfalfa meal and therefore contains minimal phytoestrogens. Animals were treated in accordance with the National Institutes of Health Guide for the Care and Use of Laboratory Animals and the established institutional animal use and care protocol at Northwestern University.

Alginate Hydrogel Preparation

Sodium alginate (55-65% guluronic acid) was provided by FMC BioPolymer (Philadelphia, Pa.) Alginate dissolved in deionized water to a concentration of 1% (w/v) and then purified with activated charcoal (0.5 g charcoal/g alginate) to remove organic impurities and improve the purity of the alginate. Following charcoal treatment, alginate solution was sterile-filtered through 0.22-μm filters, lyophilized within Steriflip conical tubes (Millipore, Billerica, Mass.), and sterile-aliquoted. Aliquots of charcoal-stripped and sterilized sodium alginate were reconstituted with sterile 1× phosphate-buffered saline (PBS) to a concentration of 1.5% (w/v) for each experiment.

Follicle Isolation, Encapsulation, and Culture

Multilayered secondary follicles (150-180 um, type 5b) were isolated from 16-day-old female mice and encapsulated into a sterile 1.5% (w/v) alginate bead as described previously with slight modifications. Ovaries were incubated in αMEM (Invitrogen, Carlsbad, Calif.) containing 1% fetal calf serum (FCS) (Invitrogen), 0.1% type I collagenase, and 0.02% DNase I (Worthington Biochemical, Lakewood, N.J.) at 37° C. and 5% carbon dioxide (CO₂) for 30 min. Follicles were mechanically isolated using insulin-gauge needles in L15 media (Invitrogen) containing 1% FCS. Individual follicles were maintained in αMEM/1% FCS at 37° C., 5% CO₂ for 2 h before encapsulation. Only follicles displaying the following characteristics during the 2-h preincubation period were selected for encapsulation and culture: (1) diameter of 150-180 um; (2) intact with some attached, fibroblast-like theca cells; and (3) visible, immature oocyte that was round and centrally located within the follicle. After washing through 1.5% alginate twice, single follicles were pipetted into the middle of each alginate droplet (2-3 μl) suspended on a polypropylene mesh (0.1-mm opening; McMaster-Carr, Atlanta, Ga.). The mesh was immediately immersed in sterile 50 mM calcium chloride for 2 min to crosslink the alginate; it was then rinsed in culture media (αMEM, 10 mIU/mL recombinant follicle-stimulating hormone [A. F. Parlow, National Hormone and Pituitary Program, National Institute of Diabetes and Digestive and Kidney Diseases, Bethesda, Md.], 3 mg/mL bovine serum albumin [BSA], 1 mg/mL bovine fetuin [Sigma-Aldrich, St. Louis, Mo.)], 5 μg/mL insulin, 5 μg/mL transferrin, and 5 ng/mL selenium). Alginate beads containing a single follicle were plated at 1 follicle per well in 96-well plates in 100 μl of culture media. Fetuin, dialyzed extensively against embryo culture-grade water and lyophilized, was added to prevent zona pellucida hardening. Throughout isolation, encapsulation, and plating, follicles were maintained at 37° C. and a pH of 7.0. Encapsulated follicles were cultured at 37° C. in 5% CO₂ for 8 days. Every other day, half of the media (50 uL) was exchanged and stored at −80° C. Follicle survival and diameter were assessed using an inverted Leica DM IRB microscope with transmitted light and phase objectives (Leica, Bannockburn, Ill.). Follicles were designated dead if the oocyte was no longer surrounded by a granulosa cell layer or if the granulosa cells had become dark and fragmented. After 8 days, the culture media was replaced by 100 μL L15 medium containing 10 mIU/mL alginate lyase for 30 min at 37° C. Follicles wee removed from the degraded alginate bead, and all remaining alginate removed using a new IVF dish containing L15 medium with 1% FCS.

Follicle and Oocyte Measurement

Pictures of encapsulated follicles were taken on culture days 0, 4, and 8 using an inverted Leica DM IRB microscope. The diameter of follicles containing oocytes that had not yet matured was measured in duplicate from the outer layer of theca cells using Image J 1.33U (National Institutes of Health, Bethesda, Md.) and was based on a calibrated ocular micrometer. The diameters of oocytes from follicles cultured in vitro were obtained on day 0 and day 8 and were compared with those of control in vivo oocytes collected from unexpanded cumulus oocyte complexes of antral follicles from superovulated 24-day-old mice (primed with 51U equine chorionic gonadotropin (eCG) [Sigma-Aldrich] for 46 h). Control oocytes were denuded by gentle aspiration through glass pipettes. The oocyte diameter was measured without the zona pellucida.

Alginate hydrogel-embedded follicles (n=129) maintained their 3D structures and had a survival rate of 93.3%+/−1.6% through an 8-day culture period. The average follicle diameter increased from 156.1+/−6.0 μm on day 0 to 348.8+/−44.8 μm on day 8. Follicles grown in vitro maintained structures that phenocopied those of in vivo control follicles: a spherical shape with a central fluid-filled antral cavity containing an oocyte surrounded by cumulus cells. Embedded follicles also had an intact theca cell layer, as revealed by 3βHSD staining.

Oocyte Maturation

After follicles were retrieved from the alginate bead, they were transferred to maturation media composed of αMEM, 10% FCS, 1.5 IU/mL human chorionic gonado-tropin, (HCG) and 5 ng/mL epidermal growth factor (Sigma-Aldrich) for 16 h at 37° C., 5% CO₂. Oocytes were then denuded from the surrounding cumulus cells by treatment with 0.3% hyaluronidase and gentle aspiration through a polished drawn glass pipette. The oocytes were considered to be in metaphase I if neither the germinal vesicle nor the first polar body was visible. If a polar body was present in the perivitelline space, the oocytes were classified as metaphase II. Fragmented or shrunken oocytes were classified as degenerated and were discarded. Control in vivo oocytes were collected from 24-day-old mice primed with eCG for 46 h, placed in maturation media, denuded, and classified as described previously.

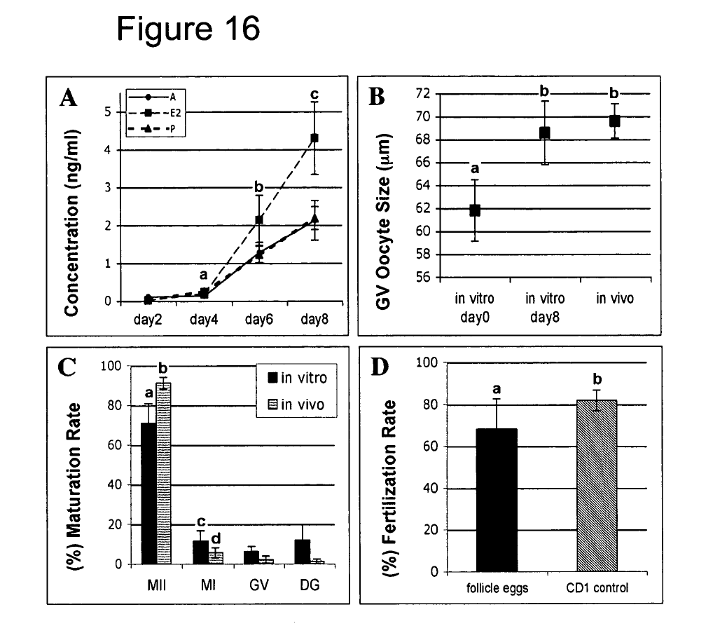

In vivo, immature oocytes grow in size while remaining in prophase I, and must undergo a process of maturation in which the germ cell progresses from prophase I to metaphase II in response to increasing concentrations of gonadotropins in order to become competent for fertilization. Similarly, oocytes cultured in vitro must mature and progress to metaphase II in response to exogenous gonadotropins, a process termed in vitro maturation. Throughout the culture period, oocytes underwent extensive growth and maintained meiotic arrest. The average size of oocytes increased from 61.78±2.67 um on day 1 to 68.57±2.77 urn on day 8 of culture (n−30; p<0.05). The diameter of oocytes grown in vitro approached that of in vivo control oocytes of the same chronologic age (69.58±1.50 urn); this difference was not statistically significant (n=30; p=0.078) (FIG. 16B).

After retrieval of the follicles from the alginate hydrogel matrix on day 8, in vitro maturation was induced by exposing the follicles to exogenous HCG, and the granulosa cells were removed. Of 99 fully grown, denuded oocytes retrieved from the alginate culture system, a mean of 82.3%±8.8% resumed meiosis and underwent germinal vesicle breakdown, 70.9%±9.9% extruded the first polar body and matured to metaphase II, and 11.4%±5.3% remained in metaphase I (FIG. 16C). Notably, the maturation rate of cultured oocytes was lower than that of control oocytes that developed in vivo, with a mean of 96.7%±0.5% undergoing germinal vesicle breakdown and 91.9%±2.9% reaching metaphase II (FIG. 16C).

IVF and Embryo Transfer

Two hours before IVF, motile sperm was prepared from a sperm suspension collected from the cauda epididymis of proven CD1 male breeder mice using Percoll gradient-centrifugation (PGC) as described elsewhere. ²⁰PGC sperm was capacitated in IVF medium (KSOM, Specialty Media, Phillipsburg, N.J.) supplemented with 3 mg/mL BSA, 5.36 mM D-glucose) for 30 min. Fifteen to 20 metaphase II oocytes were placed in 50 uL IVF medium microdrops containing 1×10⁶ sperm/mL and incubated under mineral oil for 7-8 h at 37° C., 5% CO₂. Oocytes were then washed 3 times in fresh KSOM to remove all bound sperm. Fertilized oocytes were identified by the presence of 2 pronuclei (2PN). As a control, GDI oocytes were obtained from day-24 mice primed with 5 IU of eCG for 48 h and 5 IU of HCG for 14 h before collection. The 2PN zygotes were transferred to the oviducts of 8- to 10-week-old pseudopregnant CD1 female rats 0.5 days postcoitum.

Subsequent IVF of mature oocytes should result in the extrusion of the second polar body and the formation of 2PN. In vitro-cultured, denuded oocytes in metaphase II (n=86) and control oocytes collected from superovulated mice (in vivo controls, n=65) were fertilized in vitro under the same conditions. The development of 2PN zygotes was scored as a successful fertilization, and occurred in a mean of 68.2%±14.5% of oocytes cultured in vitro and 81.7%±5.0% of in vivo control oocytes (FIG. 16D). Twenty 2PN-stage zygotes derived from oocytes cultured in vitro and 16 derived from in vivo control oocytes were transferred to the oviducts of pseudopregnant CD1 female rats, 6 zygotes per oviduct. Two female and 2 male brown pups derived from oocytes cultured in vitro (from C57BL/6×CBA F1 hybrids) and 4 (2 males/2 females) white pups derived from in vivo control oocytes (from superovulated CD1 mice) were successfully delivered after a 19-day gestation. All 4 of the mice derived from oocytes cultured in vitro developed normally and were fertile.

Histology and Theca Cell Staining

Follicles cultured for 8 days were removed from the alginate bead as described previously and fixed for 2 h at 4° C. in 4% paraformaldehyde in 1×PBS. Follicles were dehydrated in ascending concentrations of ethanol (10-100%), and embedded in paraffin by an automated tissue processor (Leica, Mannheim, Germany). Serial 4-um sections were cut and stained with hematoxylin and eosin. To verify the presence of an intact theca cell layer, follicles were stained with 3p-hydroxysteroid dehydrogenase (3PHSD) solution containing 0.12 mg/mL nitroblue tetrazolium chloride, 0.25 mg/mL p-isocitrate dehydrogenase 3-f, and 0.025 mg/mL epiandrosterone (Sigma-Aldrich) in 1×PBS for 30 min at room temperature in the dark.

Hormone Assays

Androstenedione, 17β-estradiol, and progesterone were measured in conditioned media collected on follicle culture days 2, 4, 6, and 8 using commercially available radio-immunoassay kits (androstenedione and 17β-estradiol, Diagnostic Systems Laboratories, Inc., Webster, Tex.; progesterone, Diagnostic Products Corp., Los Angeles, Calif.). Media collected from wells containing no follicle was used as the assay control.

Secretion of androstenedione, estradiol, and progesterone from follicles cultured in vitro is depicted in FIG. 16. Steroid levels were undetectable on day 2 of culture but began to increase by day 4. Mean androstenedione levels rose to 1.27+/−0.27 ng/mL at day 6 and 2.12+/−0.52 ng/mL at day 8, indicating that theca cells were exhibiting normal physiologic function in culture. Average progesterone concentrations remained under 1 ng/mL up to day 4 of culture, then increased to approximately 2 ng/mL as luteinization of granulosa cells occurred. Average estradiol production increased quickly from a mean 0.19+/−0.09 ng/mL on day 4 to 4.29+/−0.96 ng/mL on day 8 of culture.

Statistical Analysis

Oocyte survival rate, size, and steroid productions were obtained from 6 independent cultures. Two cultures were used to measure oocyte size. The other 4 cultures are for in vitro maturation, IVF, and embryo transfers. Follicle size and steroid hormone concentrations were analyzed by 1-way analysis of variance (ANOVA). Oocyte size, in vitro maturation rate, and IVF rate were analyzed using a 1-way ANOVA followed by a paired M test. A p value less than 0.05 was considered statistically significant. All statistical calculations were done with GraphPad Prism software, version 4.00 (San Diego, Calif.).

### EXAMPLE 25

### Stem Cell Generation from GOC Complexes Matured in Alginate

Cumulus cells surrounding the ooctyes can be used as stem cells for the generation of any cell type. These cumulus cells are harvested from the oocyte by treatment of the oocyte-cumulus cell complex with follicle stimulating hormone (the natural agonist) or hyluronidase (an enzyme) or a natural local ligand (such as GDF-9).

The cumulus cell DNA can then be injected into mature oocytes for the purpose of cloning. In addition, the matured oocyte can be fertilized with sperm and individual blastomeres isolated by manual dissection or enzymatic digestion. The individual blastomeres can then be used as totipotent cells for the generation of any cell type.

The source of stem cells may be a single cell such as a fertilized oocyte, or it may comprise a mixture of cells, such as cells derived from an embryo, blood or somatic tissue of a normally bred or transgenic animal or cell line. In the latter case the selectable marker may be incorporated into the transgenic animal's genome.

Researchers have isolated several key sources of stem cells. These sources include: Blastocysts (embryos after six days of growth); early embryos created by human cloning; fetal tissue; adult or child tissue; and adult or child cells that can be grown into stem cells.

Stem cells, which scientists have successfully extracted from both embryos and fetuses, represent cells that have not yet committed to a particular tissue, but, depending on what stage of growth/maturation they are in, they may be capable of evolving into a potential multitude of tissues.

Stem cells taken from adults or children (known generically as “adult stem cells”) have been used extensively and effectively in the treatment of degenerative diseases. For example, doctors at the Necker Hospital for Sick Children in Paris succeeded in treating two infants diagnosed with Severe Combined Immunodeficiency Disease (SCID), a life-threatening degenerative disease caused by defects on the male (X) chromosome. The team extracted “adult” stem cells from the children's bone marrow, manipulated the cells in the laboratory to replace the damaged gene with a functioning gene, then re-injected the cells back into the bone marrow. The repaired cells then “replenished” the immune system and the children have since gone on to make a full recovery. “Gene Therapy of Severe Combined Immunodeficiency (SCID)-X1 Disease”, Science 288, 669-672, Apr. 28, 2000.

Using a technique called “altered nuclear transfer,” R. Jaenisch has created an embryo-like entity that is genetically incapable of implantation into a uterus. Although this entity was not a viable embryo, it yielded perfectly healthy embryonic stem cells. This technique was based upon a mouse model, wherein Dr. Jaenisch demonstrated that it is possible to procure embryonic stem cells without harming a viable embryo. See Nature, 2005, Oct. 16, Generation of nuclear transfer-derived pluripotent ES cells from cloned Cdx2-deficient blastocysts, which is incorporated by reference herein in its entirety.

The technology described herein can be used in a variety of applications, including:
1) Creation of lines of personalized stem cells, either through in-vitro fertilization or somatic cell nuclear transfer.
2) Follicle, cortical tissue, or germ cell banking: we enable the usefulness of storing immature reproductive cells/tissue until it is needed later in a woman's life for pregnancy or stem cell applications.

3) Determining the effect of molecules/compounds on follicular development

TABLE 1
Follicle survival and size increase for two-layered secondary follicles
cultured in alginate-collagen I matrices and multilayered secondary
follicles cultured in alginate matrices.

| | Two-Layered | | | Multilayered | | |
|---|---|---|---|---|---|---|
| | Secondary Follicles | | | Secondary Follicles | | |
| | | | Size Increase | | | Size Increase |
| FSH Dose (mIU/mL) | n | Survival | Avg +/− SEM | n | Survival | Avg +/− SEM |
| 0 | 102 | 77.4% | 6.0 +/− 1.6% | 55 | 41.8% | 19.0 +/− 1.8% |
| 5 | 99 | 75.8% | 18.1 +/− 1.8% | 50 | 72.0% | 42.7 +/− 1.0% |
| 10 | 96 | 75.0% | 28.5 +/− 2.2% | 107 | 69.2% | 62.2 +/− 2.9% |
| 25 | 98 | 66.3% | 41.4 +/− 3.7% | 62 | 56.5% | 54.3 +/− 2.2% |
| 50 | — | — | — | 63 | 30.2% | 86.6 +/− 6.6% |

TABLE 2
Oocyte meiotic competence for two-layered secondary follicles cultured in alginate-
collagen I matrices and multilayered secondary follicles cultured in alginate matrices.

| FSH | Two-Layered Secondary Follicles | | | | Multilayered Secondary Follicles | | | | |
|---|---|---|---|---|---|---|---|---|---|
| Dose mIU/mL | n | DG % | GV % | GVBD % | n | DG % | GV % | GVBD % | PB % |
| 0 | 21 | 23.8 | 61.9 | 14.3 | 13 | 84.6 | 0.0 | 15.4 | 0.0 |
| 5 | 28 | 17.9 | 60.7 | 21.4 | 32 | 12.5 | 0.0 | 9.4 | 78.1 |
| 10 | 44 | 36.4 | 36.4 | 27.3 | 40 | 15.0 | 7.5 | 37.5 | 40.0 |
| 25 | 39 | 33.3 | 43.6 | 23.1 | 30 | 10.0 | 0.0 | 26.7 | 63.3 |
| 50 | — | — | — | — | 14 | 14.3 | 0.0 | 42.9 | 42.9 |
| IVM | 72 | 0.0 | 0.0 | 100.0 | 129 | 0.0 | 0.0 | 6.2 | 93.8 |
| IVO | — | — | — | — | 207 | 2.9 | 0.0 | 39.1 | 58.0 |

DG = degenerated,
GV = Germinal vesicle stage,
GVBD = germinal vesicle breakdown, and
PB = polar body.
IVM = in vitro matured control
IVO = in vivo ovulated control

TABLE 3
Survival and follicle size measurements for preantral two-layered secondary
follicles. Meiotic competence for two-layered secondary follicles.

| | | | Oocyte Stage After Maturation | | |
|---|---|---|---|---|---|
| | Survival % | Follicle Size Increase | Degenerated | GV | GVBD % |
| Matrix | (Starting n) | (Avg ± SEM) | % (n) | % (n) | (n) |
| Alginate | 63.9%^{a} | −0.6 ± 1.2%^{a} | 31.4%^{a} | 57.1% | 11.5 |
| | (108) | | (11) | (20) | (4) |
| Collagen 1 | 64.8%^{a} | 15.4 ± 1.6%^{b} | 46.4%^{a} | 35.7% | 17.9% |
| | (125) | | (13) | (10) | (5) |
| Fibronectin | 69.5%^{a} | 2.9 ± 1.5%^{a,c} | 48.4%^{a} | 29.0% | 22.6 |
| | (118) | | (15) | (9) | (7) |
| ROD | 71.7%^{a} | 8.8 ± 2.3%^{b,c} | 3.3%^{a} | 53.3% | 13.3% |
| | (46) | | (5) | (8) | (2) |
| Collagen IV | 72.0%^{a} | 1.3 ± 2.2%^{a,o} | 32.0%^{a} | 48.0% | 20.0 |
| | (50) | | (8) | (12) | (5) |
| Laminin | 62.5%^{a} | 1.7 ± 1.8%^{′″C} | 41.2%^{a} | 29.4% | 29.4% |
| | (64) | | (7) | (5) | (5) |

Significant differences are denoted by different superscripts, p < 0.05.
GV = germinal vesicle stage,
GVBD = germinal vesicle breakdown.

TABLE 4
Survival and follicle size measurements for preantral multilayered follicles.
Meiotic competence for multilayered secondary follicles.

| | | Follicle | Oocyte Stage After Maturation | | | |
|---|---|---|---|---|---|---|
| | Survival % | Size Increase | Degenerated | GV | GVBD % | PB |
| Matrix | (Starting n) | (Avg + SEM) | % (n) | % (n) | (n) | % (n) |
| Alginate | 69.2%^{a} | 62.2 + 2.9%^{a} | 15.0%^{a} | 7.5% | 37.5% | 40.0%^{a} |
| | (107) | | (6) | (3) | (15) | (16) |
| Collagen 1 | 67.4%^{a} | 61.2 + 4.2%^{a,b} | 25.0%^{a} | 6.3% | 25.0% | 43.8%^{a} |
| | (46) | | (4) | 0) | (4) | (7) |
| Fibronectin | 71.8%^{a} | 42.3 + 2.1%^{o} | 8.3%^{a} | 4.2% | 16.7% | 70.8%^{b} |
| | (39) | | (2) | (1) | (4) | (17) |
| RGD | 62.0%^{a} | 55.7 + 3.4%^{a,c} | 0.0%^{a} | 0.0% | 35.5% | 64.5%^{b} |
| | (71) | | (0) | (0) | (11) | (20) |
| Collagen IV | 48.1%^{a} | 46.4 + 2.8%′″^{o} | 9.1%^{a} | 4.5% | 36.4% | 50.0%^{a} |
| | (54) | | (2) | (1) | (8) | (11) |
| Laminin | 60.9%^{a} | 44.3 + 2.8%^{o} | 17.1% | 0.0% | 11.4% | 71.4%^{b} |
| | (64) | | (6) | (0) | (4) | (25) |

Significant differences are denoted by different superscripts, p < 0.05.
GV = germinal vesicle stage,
GVBD = germinal vesicle breakdown, and
PB = polar body stage.

TABLE 5
Survival rates, follicle size measurement, antrum and theca cell layer observed
rates from two-layered secondary follicles cultured in alginate scaffold in vitro.

| | | | Follicle | | | | Theca Layer | |
|---|---|---|---|---|---|---|---|---|
| | | | Diameter (μm)* | | | | (%)* | |
| Alginate Con. | N | Survival (%)* | Day 8 | Day 10 | Day 12 | Antrum (%)* | Day 8 | Day 10 |
| 1.50% | 101 | 73.6 ± 9.3 | 182.7 ± 6.3 | 193.5 ± 6.9 | 199.4 ± 8.1 | 5.0 ± 1.8^{a} | 0^{a} | 0^{a} |
| 1.00% | 108 | 84.0 ± 5.3 | 207.9 ± 8.3 | 238.5 ± 10.1 | 257.3 ± 11.9 | 63.9 ± 1.7^{b} | 14.0 ± 4.1^{b} | 18.9 ± 4.1^{b} |
| 0.50% | 118 | 84.8 ± 5.8 | 203.7 ± 6.7 | 261.6 ± 8.3 | 311.5 ± 8.7 | 73.2 ± 8.5^{c} | 66.4 ± 5.7^{c} | 79.2 ± 2.1^{c,d} |
| 0.25% | 96 | 78.0 ± 3.8 | 208.5 ± 6.0 | 274.7 ± 7.0 | 326.2 ± 6.3 | 86.2 ± 8.0^{c} | 72.2 ± 3.5^{c} | 79.3 ± 4.8^{c} |

Different letters within each column indicate statistically significant differences (p < 0.05).
Con. = concentration;
N = starting follicle number.
*Values are the average ± SEM of multiple follicles from four independent cultures.

TABLE 6
Meiotic competence, fertilization rate and developmental competence of oocytes
from two-layered secondary follicles cultured in alginate.

| Alginate Con. | N | MII* | GVBD | GV | DG | 2-cell embryos^{†} | Blstocysts^{‡} |
|---|---|---|---|---|---|---|---|
| 1.50% | 76 | 56.3%^{a} | 84.2% | 5.3% | 10.5% | 5.6%^{a} | 0.0%^{a} |
| 1.00% | 92 | 58.3%^{a} | 78.3% | 10.9% | 10.9% | 14.3%^{a} | 0.0%^{a} |
| 0.50% | 95 | 67.1%^{b} | 86.3% | 6.3% | 7.4% | 11.5%^{b} | 0.0%^{b} |
| 0.25% | 76 | 67.2%^{b} | 88.2% | 3.9% | 9.2% | 41.5%^{c} | 29.4%^{b} |

Different letters within each column indicate statistically significant differences (p < 0.05).
Con. = concentration;
N = surviving follicle number;
MII = metaphase II;
GVBD = germinal vesicle breakdown;
GV = germinal vesicle;
DG = degenerate.
*The percent of MII oocytes was calculated as a proportion of oocytes undergoing GVBD.
^{†}2-cell embryos/MII oocytes
^{‡}Day 5 blastocysts/2-cell

While the principals of this invention have been described in connection with specific embodiments, it should be understood clearly that these descriptions are added only by way of example and are not intended to limit, in any way, the scope of this invention. For instance, the present invention can be utilized in conjunction with growth or maturation systems including a variety of 3-dimensional polymeric matrix materials, including suitable coupling or cross-linking agents or structural moieties. Other advantages and features of this invention will become apparent from the claims hereinafter, with the scope of those claims determined by their reasonable equivalents, as would be understood by those skilled in the art.

We claim:

1. An in vitro method for maturing a two-layered secondary follicle comprising:
(a) suspending a two-layered secondary follicle into a non-crosslinked droplet of an alginate-ECM solution, wherein the solution contains less than 2% alginate weight per volume;
(b) crosslinking the suspension-solution, thereby forming a two-layered secondary follicle-three dimensional alginate-ECM gel matrix;
(c) culturing the two-layered secondary follicle in the three dimensional matrix, wherein the two-layered secondary follicle develops into a multilayered secondary follicle having more than two layers of granulosa cells; and
(d) releasing the multilayered secondary follicle from the three dimensional gel matrix.

2. The method of claim 1, wherein the alginate-ECM solution comprises alginate in a weight per volume percentage selected from the group consisting of 1.5%, 1.0%, 0.75%, 0.5%, and 0.25%.

3. The method of claim 1, wherein the alginate-ECM gel matrix comprises ECM proteins selected from the group consisting of collagen I, fibronectin, collagen IV, laminin, peptides comprising an RGD amino acid sequence, the peptide IKVAV, and the peptide YIGSR.

4. The method of claim 1, wherein the two-layered secondary follicle is a type 4 follicle (100-130 μm).

5. The method of claim 3, wherein the alginate-ECM matrix comprises the ECM proteins collagen I and peptides having the RGD amino acid sequence.

6. The method of claim 5, wherein peptides having an RGD amino acid sequence is selected from the group consisting of GGGGRGDS, GRGDY, GGGGRGD, and the RGD tri-peptide.

7. An in vitro method for maturing a multi-layered secondary follicle having more than two layers of granulosa cells comprising:
(a) suspending a multi-layered secondary follicle having more than two layer of granulosa cells into a non-crosslinked droplet of an alginate solution, wherein the solution contains less than 2% alginate weight per volume;
(b) crosslinking the suspension, thereby forming a multi-layered secondary follicle-three dimensional alginate gel matrix;
(c) culturing the multi-layered secondary follicle in the three dimensional matrix in an FSH solution, wherein the multi-layered secondary follicle develops an antral cavity; and
(d) releasing the multi-layered secondary follicle comprising the antral cavity from the three dimensional gel matrix.

8. The method of claim 7, wherein the multilayered secondary follicles are type 5b (150-180 μm).

9. The method of claim 7, wherein step (c) further comprises culturing the multi-layered secondary follicle in the three dimensional matrix for about 12 days.

10. The method of claim 7 further comprising:
(e) transferring the multi-layered secondary follicle to maturation media;
(f) denuding the oocytes from surrounding cumulus cells; and
(g) scoring for extrusion of polar body.

11. The method of claim 7, wherein step (a) further comprises one or more ECM proteins.

12. The method of claim 7, wherein the FSH solution contains between 1 mIU/ml and 50 mIU/ml of FSH.

13. The method of claim 12, wherein the FSH solution contains 5 mIU/ml of FSH.

14. The method of claim 12, wherein the FSH solution contains 10 mIU/ml of FSH.

15. The method of claim 12, wherein the FSH solution contains 15 mIU/ml of FSH.

16. The method of claim 12, wherein the FSH solution contains 20 mIU/ml of FSH.

17. The method of claim 12, wherein the FSH solution contains 25 mIU/ml of FSH.

18. An in vitro follicle cell maturation kit, comprising (a) a first container means containing a crosslinkable alginate solution having less than 2% alginate weight per volume; (b) a second container means containing a crosslinking agent; (c) a third container means containing one or more growth factors; (d) a fourth container containing one or more peptides comprising an RGD amino acid sequence; and (e) a fifth container containing alginate lyase.

19. The method of 18, wherein the crosslinking agent is calcium chloride (CaCl₂).

20. An in vitro method for maturing a preantral follicle comprising:
(a) suspending a preantral follicle into a non-crosslinked alginate solution, wherein the solution comprises less than 2% alginate weight per volume;
(b) crosslinking the suspension, thereby forming a preantral follicle-three dimensional gel matrix;
(c) culturing the preantral follicle in the three dimensional matrix, wherein the preantral follicle forms an antral cavity and whereby a cumulus-oocyte complex is formed; and
(d) releasing the antral follicle from the three dimensional gel matrix.

21. The method of claim 20 further comprising:
(e) culturing the released antral follicle in culture media comprising one or more pituitary hormones, wherein polar bodies are formed; and
(f) releasing the oocyte from the antral follicle.

22. The method of claim 20 further comprising:
(e) isolating the cumulus-oocyte complex from the antral follicle;
(f) culturing the isolated cumulus-oocyte complex in culture media comprising one or more pituitary hormones, wherein polar bodies are formed; and
(g) releasing the oocyte from the cumulus-oocyte complex.

23. The method of claim 20 further comprising:
(e) isolating the cumulus-oocyte complex from the antral follicle;
(f) culturing the isolated cumulus-oocyte complex in culture media comprising one or more pituitary hormones, wherein polar bodies are formed; and
(g) removing the cumulus-oocyte complex from the culture.

24. The method of claim 20 further comprising:
(e) culturing the released antral follicle in culture media comprising one or more pituitary hormones, wherein polar bodies are formed; and
(f) isolating the cumulus oocyte complex from the cultured antral follicle.

25. The method of claim 20, wherein the three dimensional gel matrix comprises a polysaccharide.

26. The method of claim 25, wherein the polysaccharide is selected from the group consisting of alginate and hyaluronic acid.

27. The method of claim 20, wherein one or more growth factors are added to the non-crosslinked solution.

28. The method of claim 27, wherein the one or more growth factors are selected from the group consisting of inhibins, activins, selenites, and transferring.

29. The method of claim 20, wherein one or more hormones are added to the non-crosslinked solution.

30. The method of claim 29, wherein the hormones are selected from the group consisting of follicle stimulating hormone and luteinizing hormone.

31. The method of claim 20, wherein the preantral follicle is mammalian.

32. The method of claim 31, wherein the mammalian preantral follicle is human.

33. The method of claim 31, wherein the mammalian preantral follicle is murine.

34. The method of claim 10, wherein the maturation media comprises αMEM, 10% FCS, 1.5 IU/ml hCG and 5 ng/ml EGF for about 16 hours at 37° C., 5% CO₂.

35. A method for the in vitro production of a stem cell line comprising the steps of:
(a) suspending a preantral follicle into a non-crosslinked alginate solution, wherein the solution comprises less than 2% alginate weight per volume; (b) crosslinking the suspension, thereby forming a preantral follicle-three dimensional gel matrix; (c) culturing the preantral follicle-three dimensional gel matrix in culture containing one or more pituitary hormones, wherein the preantral follicle forms an antral cavity and whereby a cumulus-oocyte complex is formed; (d) releasing the antral follicle from the three dimensional gel matrix; (e) culturing the released antral follicle in culture media comprising one or more pituitary hormones, wherein polar bodies are formed; (f) releasing the oocyte from the antral follicle; (g) fertilizing the oocyte in vitro, thereby forming a preimplantation embryo; (h) culturing the resultant embryo in vitro, wherein a blastocyst is formed; and (i) deriving stem cells derived from the blastocyst.

36. The method of claim 35, wherein the one or more pituitary hormones in step (c) is follicle stimulating hormone.

37. The method of claim 35, wherein the one or more pituitary hormones in step (e) is selected from the group consisting of luteinizing hormone and human chorionic gonadotropin.

38. A method for the in vitro production of a stem cell line comprising the steps of:
(a) suspending a preantral follicle into a non-crosslinked alginate solution, wherein the solution comprises less than 2% alginate weight per volume; (b) crosslinking the suspension, thereby forming a preantral follicle-three dimensional gel matrix; (c) culturing the preantral follicle-three dimensional gel matrix in culture containing one or more pituitary hormones for about 8 hours; (d) releasing the follicle from the three dimensional gel matrix; (e) culturing the released follicle in culture media comprising one or more pituitary hormones, wherein polar bodies are formed; (f) releasing the oocyte from the follicle; (g) fertilizing the oocyte in vitro, thereby forming a preimplantation embryo; (h) culturing the resultant embryo in vitro, wherein a blastocyst is formed; and (i) deriving stem cells derived from the blastocyst.

39. The method of claim 38, wherein the one or more pituitary hormones in step (c) is follicle stimulating hormone.

40. The method of claim 38, wherein the one or more pituitary hormones in step (e) is selected from the group consisting of luteinizing hormone and human chorionic gonadotropin.

41. A method for the in vitro production of a stem cell line comprising the steps of:
(a) suspending a preantral follicle into a non-crosslinked alginate solution, wherein the solution comprises less than 2% alginate weight per volume; (b) crosslinking the suspension, thereby forming a preantral follicle-three dimensional gel matrix; (c) culturing the preantral follicle-three dimensional gel matrix in culture containing one or more pituitary hormones, wherein a mature follicle is formed; (d) releasing the mature follicle from the three dimensional gel matrix; (e) culturing the released mature follicle in culture media comprising one or more pituitary hormones, wherein polar bodies are formed; (f) releasing the oocyte from the mature follicle; (g) fertilizing the oocyte in vitro, thereby forming a preimplantation embryo; (h) culturing the resultant embryo in vitro, wherein a blastocyst is formed; and (i) deriving stem cells derived from the blastocyst.

42. The method of claim 41, wherein the one or more pituitary hormones in step (c) is follicle stimulating hormone.

43. The method of claim 31, wherein the one or more pituitary hormones in step (e) is selected from the group consisting of luteinizing hormone and human chorionic gonadotropin.

44. The method according to claims 35, 38, or 41, wherein the three dimensional gel matrix comprises a polysaccharide.

45. The method according to claim 44, wherein the polysaccharide is selected from the group consisting of alginate and hyaluronic acid.

46. The method according to claims 35, 38, or 41, wherein one or more growth factors are added to the non-crosslinked solution.

47. The method of claim 46, wherein the one or more growth factors are selected from the group consisting of inhibins, activins, selenites, and transferring.

48. The method according to claims 35, 38, or 41, wherein the preantral follicle is mammalian.

49. The method of claim 48, wherein the mammalian preantral follicle is human.

50. The method of claim 48, wherein the mammalian preantral follicle is murine.
